(19) United States
(12) Patent Application Publication
(10) Pub. No.: US 2021/0383924 A1
(43) Pub. Date: Dec. 9, 2021

(54) METHODS AND MACHINE LEARNING FOR DISEASE DIAGNOSIS

(71) Applicants: QUADRANT BIOSCIENCES INC., Syracuse, NY (US); THE RESEARCH FOUNDATION FOR THE STATE UNIVERSITY OF NEW YORK, Syracuse, NY (US); THE PENN STATE RESEARCH FOUNDATION, Syracuse, NY (US)

(72) Inventors: Alexander RAJAN, Baldwinsville, NY (US); Steven D. HICKS, Hershey, PA (US); Frank A. MIDDLETON, Fayetteville, NY (US)

(73) Assignees: QUADRANT BIOSCIENCES INC., Syracuse, NY (US); THE RESEARCH FOUNDATION FOR THE STATE UNIVERSITY OF NEW YORK, Syracuse, NY (US); THE PENN STATE RESEARCH FOUNDATION, University Park, PA (US)

(21) Appl. No.: 17/288,399

(22) PCT Filed: Oct. 25, 2019
(86) PCT No.: PCT/US2019/058073

§ 371 Date: Apr. 23, 2021

Related U.S. Application Data
(60) Provisional application No. 62/816,328, filed on Mar. 11, 2019.
(60) Provisional application No. 62/750,401, filed on Oct. 25, 2018.
(60) Provisional application No. 62/750,378, filed on Oct. 25, 2018.

(51) Int. Cl. G16H 50/20; G16H 10/60; G16H 10/40; G16H 10/20; G16B 40/00

(52) U.S. Cl. CPC G16H 50/20; G16H 10/60; G16B 40/00; G16H 10/20; G16H 10/40

(57) ABSTRACT

A machine learning classifier that diagnoses autism spectrum disorder (ASD) is described that transforms data obtained from a patient medical history and a patients saliva into data that correspond to a test panel of features, the data for the features including human microtranscriptome and microbial transcriptome data, wherein the transcriptome data are associated with respective RNA categories for ASD. The classifier classifies the transformed data by applying the data to the classifier that has been trained to detect ASD using training data associated with the features of the test panel. The trained classifier includes vectors that define a classification boundary and predicts a probability of ASD based on results of the classifying.

### CROSS-REFERENCE TO RELATED APPLICATIONS

This application is related to Provisional Patent Application Nos. 62/816,328 filed Mar. 11, 2019; 62/750,378, filed Oct. 25, 2018; 62/750,401, tiled Oct. 25, 2018; 62/474,339, filed Mar. 21, 2017; 62/484,357, filed Apr. 11, 2017; 62/484,332, filed Apr. 11, 2017; 62/502,124, filed May 5, 2017; 62/554,154, filed Sep. 5, 2017; 62/590,446, filed Nov. 24, 2017; 62/622,319, filed Jan. 26, 2018; 62/622,341, filed Jan. 26, 2018; and 62/665,056, tiled May 1, 2018, the entire contents of which are incorporated herein by reference.

This application is related to International Application Nos. PCT/US18/23336, filed Mar. 20, 2018; PCT/US18/23821, filed Mar. 22, 2018; and PCT/US18/24111, filed Mar. 23, 2018, the entire contents of which are incorporated herein by reference.

### BACKGROUND

### Field of the Disclosure

The present disclosure relates generally to a machine learning system and method that may be used, for example, diagnosing of mental disorders and diseases, including Autism Spectrum Disorder and Parkinson's Disease, or brain injuries, including Traumatic Brain Injury and Concussion.

### Description of the Related Art

Certain biological molecules are present, absent, or have different abundances in people with a particular medical condition as compared to people without the condition. These biological molecules have the potential to be used as an aid to diagnose medical conditions accurately and early in the course of development of the condition. As such, certain biological molecules are considered as a type of biomarker that can indicate the presence, absence, or degree of severity of a medical condition. Principal types of biomarkers include proteins and nucleic acids; DNA and RNA. Diagnostic tests using biomarkers require obtaining a sample of a biologic material, such as tissue or body fluid, from which the biomarkers can be extracted and quantified. Diagnostic tests that use a non-invasive sampling procedure, such as collecting saliva, are preferred over tests that require an invasive sampling procedure such as biopsy or drawing blood. RNA is an attractive candidate biomarker because certain types of RNA are secreted by cells, are present in saliva, and are accessible via non-invasive sampling.

A problem that affects use of biomarkers as diagnostic aids is that while the relative quantities of a biomarker or a set of biomarkers may differ in biologic samples between people with and without a medical condition, tests that are based on differences in quantity often are not sensitive and specific enough to be effectively used for diagnosis. In other words, the quantities of many biomarkers vary between people with and without a condition, but very few biomarkers have an established normal range which has a simple relationship with a condition, such that if a measurement of a person's biomarker is outside of the range there is a high probability that the person has the condition.

Although extensive studies have been made on biomarkers and their relationship to medical conditions, the relationships are often complex with no simple biomarker quantity range that can accurately predict with high probability that a person has a medical condition. Other factors are involved, such as environmental factors and differences in patient characteristics. Huge numbers of microorganisms inhabit the human body, especially the gastrointestinal tract, and it is known that there are many biologic interactions between a person and the population of microbes that inhabit the person's body. The species, abundance, and activity of microbes that make up the human microbiome vary between individuals for a number of reasons, including diet, geographic region, and certain medical conditions. Biomarker quantities may not only vary due to medical conditions, but may also be affected by characteristics of a patient and conditions under which samples are taken. Biomarker quantities may be affected by differences in patient characteristics, such as age, sex, body mass index, and ethnicity. Biomarker quantities may be impacted by clinical characteristics, such as time of sample collection and time since last meal. Thus, the potential number of factors that may need to be considered in order to accurately predict a medical condition may be very large.

### SUMMARY OF THE INVENTION

With a large number of possible factors to consider and no easy way of correlating the factors with a medical condition, machine learning methods have been viewed as viable techniques for medical diagnosis, Machine learning methods have been used in designing test models that are implemented in software for use in identifying patterns of information and classifying the patterns of information. However, even machine learning methods require a certain level of knowledge, such as which factors represent a medical condition and which of those factors are necessary for achieving high prediction accuracy. If a machine learning method is accurate on data it was trained on but does not accurately predict diagnosis in new patients, the model may be overfitting the training cohort and not generalize well to the general population. In order to develop a machine learning model to accurately diagnose a medical condition, a set of features that best predicts the medical condition needs to be discovered. A problem occurs, however, that the set of features that best predicts the medical condition is typically not yet known.

There is a need for a method of accurately predicting a medical condition in a patient characterized by feature values that a machine learning method has not previously seen by way of a training method that can determine a set of features that will enable prediction of the medical condition with high precision and recall.

These and other objects of the present invention will become more apparent in conjunction with the following detailed description of the preferred embodiments, either alone or in combinations thereof.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a flowchart for a method of developing a machine learning model to diagnose a target medical condition in accordance with exemplary aspects of the disclosure;

FIG. 2 is a flowchart for the data collection step of FIG. 1;

FIG. 3 is a system diagram for development and testing a machine learning model for diagnosing a medical condition in accordance with exemplary aspects of the disclosure;

FIG. 4 is a flowchart for the data transforming step of FIG. 1;

FIG. 5 is a flowchart for the feature selection and ranking step of FIG. 1;

FIG. 6 is a flowchart for the test panel selecting step of FIG. 1;

FIG. 7 is a flowchart for the test sample testing step of FIG. 1;

FIG. 8 is a diagram for a neural network architecture in accordance with an exemplary aspect of the disclosure.

FIG. 9 is a schematic for an exemplary deep learning architecture.

FIG. 10 is a schematic for a hierarchical classifier in accordance with an exemplary aspect of the disclosure.

FIG. 11 is a flowchart for developing a machine learning model for ASD in accordance with exemplary aspects of the disclosure;

FIGS. 12A, 12B, 12C is an exemplary Master Panel resulting from applying processing according to the method of FIG. 8;

FIGS. 13A, 13B, 13C, 13D is a further exemplary Master Panel resulting from applying processing according to the method of FIG. 8;

FIG. 14 is an exemplary Test Panel resulting from applying processing according to the method of FIG. 8;

FIG. 15 is a flowchart for a machine learning model for determining a probability of being affected by ASD; and

FIG. 16 is a system diagram for a computer in accordance with exemplary aspects of the disclosure.

### DETAILED DESCRIPTION

As used herein any reference to “one embodiment” or “some embodiments” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. In addition, the articles “a” and “an” as used in this application and the appended claims are to be construed to mean “one or more” or “at least one” unless specified otherwise.

The following description relates to a system and method for diagnosing a medical condition, i.n particular medical conditions related to the central nervous system and brain injury. The method optimizes the diagnostic capability of a machine learning model for the particular medical condition.

Supervised machine learning is a category of methods for developing a predictive model using labelled training examples, and once trained a machine learning model may be used to predict the disorder state of a patient using a machine learned, previously unknown function, Supervised machine learning models may be taught to learn linear and non-linear functions. The training examples are typically a set of features and a known classification of the sampled features.

From another perspective, the data itself may not be ideal. For example, photographs used for training a machine learning model may not clearly show a person's hair, or clearly distinguish a person's hair from a background. There will be noise in the data, introduced by biological or technical variation and imperfect methods. Also, there may be correlations between features: features may not be independent from one another. In such a case, highly correlated features may be removed as redundant.

As described above, features related to diagnosis of a medical condition may be extensive and the relationship between the features and condition is not as simple as a range of quantities of biological molecules that are contained in a sample. The range of quantities themselves may vary due to other environmental and patient-related factors. An objective of the present disclosure is to combine human RNA biomarkers, microbial RNA biomarkers, and patient information or health records in order to select a subset of features that improves the performance of a machine learning model. Doing so may additionally optimize the diagnostic capability of the machine learning model to aid diagnosis of patients at earlier developmental stages or stages of disease progression.

A molecular biomarker is a measurable indicator of the presence, absence, or severity of some disease state. Among types of molecules that can be used as biomarkers, RNA is an attractive candidate biomarker because certain types of RNA are secreted by cells, are present in saliva, and are accessible via non-invasive sampling. Human non-coding regulatory RNAs, oral microbiota identities (a taxonomic class, such as species, genus, or family), and RNA activity are able to provide biological information at many different levels: genomic, epigenomic, proteomic, and metabolomic.

Human non-coding regulatory RNA (ncRNA) is a functional RNA molecule. ncRNAs are considered non-coding because they are not translated into proteins. Types of human non-coding RNA include transfer RNAs (tRNAs) and ribosomal RNAs (rRNAs), as well as small RNAs such as microRNAs (miRNAs), short interfering RNAs (siRNAs), PIWI-interacting RNAs (piRNAs), small nucleolar RNAs (snoRNAs), small nuclear RNAs (snRNAs), and the long ncRNAs such as long intergenic noncoding RNAs (lincRNAS).

MicroRNAs are short non-coding RNA molecules containing 19-24 nucleotides that bind to mRNA, and silence and regulate gene expression via the binding (see Ambros et al., 2004; Bartel et al, 2004). MicroRNAs affect expression of the majority of human genes, including CLOCK, BMAL1, and other circadian genes. Each miRNA can bind to many mRNAs, and each mRNA may be targeted by several miRNAs. Notably, miRNAs are released by the cells that make them and circulate throughout the body in all extracellular fluids, where they interact with other tissues and cells. Recent evidence has shown that human miRNAs even interact with the population of bacterial cells that inhabit the lower gastrointestinal tract, termed the gut microbiome (Yuan et al., 2018). Moreover, circadian changes in miRNA abundance have recently been established (Hicks et al., 2018).

The many-to-many divergence and convergence, combined with cell-to-cell transport of miRNAs, suggests a critical systemic regulatory role for miRNAs. Nearly 70% of mi.RNAs are expressed in the brain, and their expression changes throughout neurodevelopment and varies across brain regions. Neurogenesis, synaptogenesis, neuronal migration, and memory all involve miRNAs, which are readily transported across the blood-brain-barrier. Together, these features explain why miRNA expression may be “altered” in the CNS of people with neurological disorders, and why these alterations are easily measured in peripheral biofluids, such as saliva.

A miRNA standard nomenclature system uses “miR” followed by a dash and a number, the latter often indicating order of naming. For example, miR-120 was named and likely discovered prior to miR-241. A capitalized “miR-” refers to the mature form of the miRNA, while the uncapitalized “mir-” refers to the pre-miRNA and the pri-miRNA, and “MIR” refers to the gene that encodes them. Human miRNAs are denoted with the prefix “hsa-”.

miRNA elements. Extracellular transport of miRNA via exosomes and other microvesicles and lipophilic carriers is an established epigenetic mechanism for cells to alter gene expression in nearby and distant cells. The microvesicles and carriers are extruded into the extracellular space, where they can dock and enter and the transported miRNA may then block the translation of mRNA into proteins (see Xu et al., 2012). In addition, the microvesicles and carriers are present in various bodily fluids, such as blood and saliva (see Gallo et al., 2012), enabling the measurement of epigenetic material that may have originated from the central nervous system (CNS) simply by collecting saliva. Many of the detected miRNAs in saliva may be secreted into the oral cavity via sensory nerve afferent terminals and motor nerve efferent terminals that innervate the tongue and salivary glands and thereby provide a relatively direct window to assay miRNAs which might be dysregulated in the CNS of individuals with neurological disorders.

Transfer RNA is an adaptor molecule composed of RNA, typically 76 to 90 nucleotides in length, that serves as the physical link between the mRNA and the amino acid sequence of proteins.

Ribosomal RNA is the RNA component of the ribosome, and is essential for protein synthesis.

SiRNA is a class of double-stranded RNA molecules, 20-25 base pairs in length, similar to miRNA, and operating within the RNA interference (RNAi) pathway. It interferes with the expression of specific genes with complementary nucleotide sequences by degrading mRNA after transcription, preventing translation.

piRNAs are a class of RNA molecules 26-30 nucleotides in length that form RNA-protein complexes through interactions with piwi proteins. These complexes are believed to silence transposons, methylate genes, and can be transmitted maternally. SnoRNAs are a class of small RNA molecules that primarily guide chemical modifications of other RNAs, mainly ribosomal RNAs, transfer RNAs and small nuclear RNAs. The functions of snoRNAs include modification (methylation and pseudouridylation) of ribosomal RNAs, transfer RNAs (tRNAs), and small nuclear RNAs, affecting ribosomal and cellular functions, including RNA maturation and pre-mRNA splicing. snoRNAs may also produce functional analogs to miRNAs and piRNAs.SnRNA is a class of small RNA molecules that are found within the splicing speckles and Cajal bodies of the cell nucleus in eukaryotic cells. The length of an average snRNA is approximately 150 nucleotides.

Long non-coding RNAs play roles in regulating chromatin structure, facilitating or inhibiting transcription, facilitating or inhibiting translation, and inhibiting miRNA activity.

microbiome elements. Huge numbers of microorganisms inhabit the human body, especially the gastrointestinal tract, and it is known that there are many biologic interactions between a person and the population of microbes that inhabit the person's body. The species, abundance, and activity of microbes that make up the human microbiome vary between individuals for a number of reasons, including diet, geographic region, and certain medical conditions. There is growing evidence for the role of the gut-brain axis in ASD and it has even been suggested that abnormal microbiome profiles propel fluctuations in centrally-acting neuropeptides and drive autistic behavior (see Mulle et al., 2013).

Microbial Activity. Aside from RNA and microbes, functional orthologs may be identified based on a database of molecular functions. Kyoto Encyclopedia of Genes and Genomes (KEGG) maintains a database to aid in understanding high-level functions and utilities of a biological system from molecular-level information. Molecular functions for KEGG Orthology are maintained in a database containing orthologs of experimentally characterized genes/proteins. Molecular functions in the KEGG Orthology (KO) are identified by a K number. For example, a molecule mercuric reductase is identified as K00520. A tRNA is identified as K14221. A molecule orotidine-5′-phosphate decarboxylase is identified as K01591. F-type H+/Na+-transporting ATPase subunit alpha is identified as K02111. Other tRNAs include K14225, K14232. A molecule aspartate-semialdehyde dehydrogenase is identified as K00133. A DNA binding protein is identified as K03111. These and other molecular functions have orthologs that may serve as biomarkers for medical conditions.

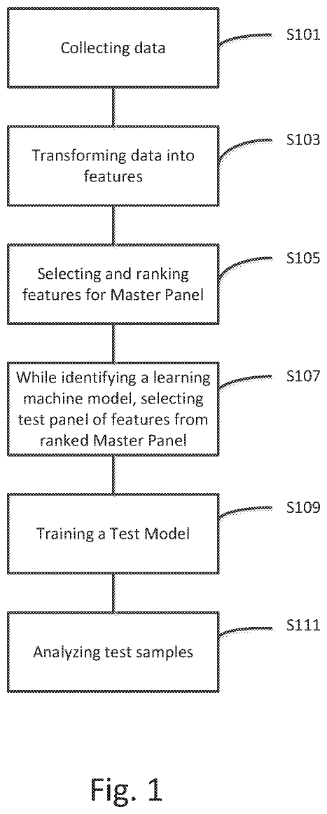

The present disclosure begins with a description of development of a machine learning model for diagnosis of a medical condition. A practical example is then provided for the embodiment of early diagnosis of Autism Spectrum disorder (ASD). FIG. 1 is a flowchart for development of a machine learning model and testing in accordance with exemplary aspects of the present disclosure. Development of a machine learning model includes data collection (S101), transforming data into features (S103), selecting and ranking features that are associated with a medical condition for a Master Panel (S105), selecting a Test Panel of features from ranked Master Panel (S107), determining a set of Test Panel features which serve as a Test Model that can be used to distinguish people with and without a target condition (S109), and analyzing test samples from patients by comparing there against the set of Test Panel features patterns that comprise the Test Model (S111).

Data collection (S101) is performed from samples obtained through a fast and non-invasive sampling, such as a saliva swab. Among other things, non-invasive sampling facilities collecting a large quantity of data required in the development of a machine learning model. For example, participants reluctant to have blood drawn will have higher compliance. Data is collected for subjects that include patients with the medical condition for which the test is to be used, healthy individuals that do not have the medical condition, and individuals with disorders that are similar to the medical condition.

Thus, the cohort for building and training a model should be as similar as possible to the intended population for the diagnostic test. For example, a diagnostic model to identify children aged 2-6 years with ASD includes subjects across the age range, with and without ASD, and with and without non-ASD developmental delays, a population which is historically difficult to differentiate from children with ASD. Likewise, to develop a diagnostic model to identify adults aged 60 to 80 with Parkinson's disease (PD), subjects preferably span the age range and include adults with PD, without PD, and with non-Parkinsonian motor disorders. Subjects are preferably sampled with a range of comorbid conditions. Further, to ensure generalizability of the diagnostic aid, subjects are preferably drawn from the range of ethnic, regional, and other variable characteristics to whom the diagnostic aid may be targeted.

The ratio of subjects with the disease/disorder to subjects without the disorder should be selected. with respect to the machine learning models to be evaluated, regardless of the disorder incidence and prevalence. For example, most types of machine learning perform best with balanced class samples. Accordingly, the class balance within the sampled subjects should be close to 1:1, rather than the prevalence of the disorder (e.g., 1:51).

Test subjects, who are not used for development of the machine learning model, should accordingly be within the ranges of characteristics from the training data. For example, a diagnostic aid for ASD in children ages 2-6 should not be applied to a 7-year-old child.

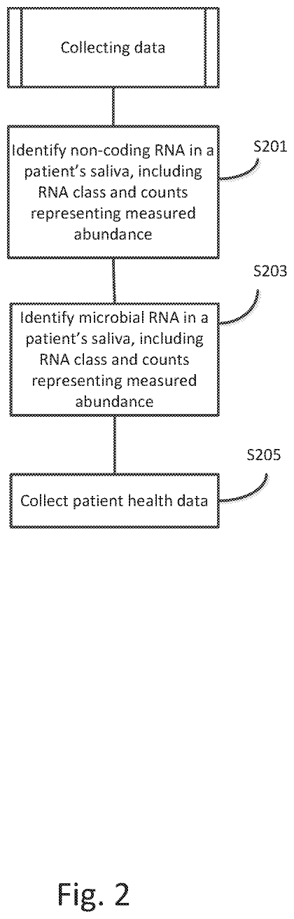

FIG. 2 is a flowchart for the data collecting of FIG. 1. In some embodiments, RNA data is collected for non-coding RNA (S201) and microbial RNA (S201). Also, patient data (S205) is collected as it relates to the patient medical history, age, and sex as well as with respect to the sampling (e.g., time of collection and time since last meal).

Data is collected from samples obtained from the subjects. In some embodiments, RNA data are derived from saliva via next generation RNA sequencing and identified using third party aligners and library databases, and categorical RNA class membership is retained. The RNA classes utilized are mature micro RNA (miRNA), precursor micro RNA (pre-miRNA), PIWI-interacting RNA (piRNA), small nucleolar RNA (snoRNA), long non-coding RNA (lncRNA), ribosomal RNA (rRNA), microbial taxa identified by RNA (microbes), and microbial gene expression (microbial activity). Together these RNAs components comprise the human microtranscriptome and microbial transcriptome. In the case of saliva samples, this is referred to as the oral transcriptome. These non-coding and microbial RNAs play key regulatory roles in cellular processes and have been implicated in both normal and disrupted neurological states, including neurodevelopmental disorders such as autism spectrum disorder (ASD), neurodegenerative diseases such as Parkinson's Disease (PD), and traumatic brain injuries (TBI).

Biomarkers may be extracted from saliva, blood, serum, cerebrospinal fluid, tissue biopsy, or other biological samples. in the one embodiment, the biological sample can be obtained by non-invasive means, in particular, a saliva sample. A swab may be used to sample whole-cell saliva and the biomarkers may be extracellular RNAs. Extracellular RNAs can be extracted from the saliva sample using existing known methods.

Optionally, saliva may be replaced by or complemented with other tissues or biofluids, including blood, blood serum, buccal sample, cerebrospinal fluid, brain tissue, and/or other tissues.

Optionally, RNA may be replaced by or complemented with metabolites or other regulatory molecules. RNA also may be replaced by or complemented with the products of the RNA, or with the biological pathways in which they participate. RNA may be replaced by or complemented with DNA, such as aneuploidy, indels, copy number variants, trinucleotide repeats, and or single nucleotide variants.

An optional second collection, of the same or other biological tissue as the first sample, may be collected at the same or different time as the original swab, to allow for replication of the results, or provide additional material if the first swab does not pass subsequent quality assurance and quantification procedures.

In one embodiment, the sample container may contain a medium to stabilize the target biomarkers to prevent degradation of the sample. For example, RNA biomarkers in saliva may be collected with a kit containing RNA stabilizer and an oral saliva swab. Stabilized saliva may be stored for transport or future processing and analysis as needed, for example to allow for batch processing of samples.

Patient data may include, but is not limited to, the following: age, sex, region, ethnicity, birth age, birth weight, perinatal complications, current weight, body mass index, oropharyngeal status (e.g. allergic rhinitis), dietary restrictions, medications, chronic medical issues, immunization status, medical allergies, early intervention services, surgical history, and family psychiatric history. Given the prevalence of attention deficit hyperactivity disorder (ADHD) and gastrointestinal (GI) disturbance among children with ASD, for purposes of the embodiment directed to ASD, survey questions were included to identify these two common medical co-morbidities. GI disturbance is defined by presence of constipation, diarrhea, abdominal pain, or reflux on parental report, ICD-10 chart review, or use of stool softeners/laxatives in the child's medication list. ADHD is defined by physician or parental report, or ICD-10 chart review.

Patient data may be collected via questionnaire completed by the patient, by the patient's parent(s) or caregiver(s), by the patient's physician, or by a trained person, and/or may be obtained from patient's medical charts. Optionally, answers collected within the questionnaire may be validated, confirmed, or made complete by the patient, patient's parent(s) or caregiver(s), or by the patient's physician.

To confirm diagnosis or lack of diagnosis for patients whose samples were used to train and test the Test Model, standard measurements of behavioral, psychological, cognitive, and medical may be performed. In the preferred embodiment of a diagnostic test for ASD in children, adaptive skills in communication, socialization, and daily living activities may be measured in all participants using the Vineland Adaptive Behavior Scale (VABS)-II. Evaluation of autism symptomology (ADOS-II) may be completed when possible for ASD and DD participants (n=164). Social affect (SA), restricted repetitive behavior (RRB) and total ADOS-II scores may be recorded. Mullen Scales of Early Learning may also be used. An example of a compilation of patient data is shown below in Table 1.

TABLE 1
Participant characteristics

| | | | | |
|---|---|---|---|---|
| Characteristic | All groups (n = 381) | ASD (n = 187) | TD (n = 125) | DD (n = 69) |

Demographics and anthropometrics

| | | | | | | | | |
|---|---|---|---|---|---|---|---|---|
| Age, months (SD) | 51 | (16) | 54 | (15) | 47 | (18)^{a} | 50 | (13) |
| Male, no. (%) | 285 | (75) | 161 | (86) | 76 | (60)^{a} | 48 | (70)^{a} |
| Caucasian, no. (%) | 274 | (72) | 132 | (71) | 95 | (76) | 47 | (69) |
| Body mass index, | 18.9 | (11) | 17.2 | (7) | 21.2 | (16) | 19.5 | (10) |
| kg/m² (SD) | | | | | | | | |

Clinical characteristics

| | | | | | | | | |
|---|---|---|---|---|---|---|---|---|
| Asthma, no. (%) | 43 | (11) | 19 | (10) | 10 | (8) | 14 | (20) |
| GI disturbance, no. | 50 | (13) | 35 | (19) | 2 | (2)^{a} | 13 | (19) |
| (%) | | | | | | | | |
| ADHD, no (%) | 74 | (19) | 43 | (23) | 10 | (8)^{a} | 21 | (30) |
| Allergic rhinitis, no. | 81 | (21) | 47 | (25) | 19 | (15) | 15 | (22) |
| (%) | | | | | | | | |

Oropharyngeal factors

| | | | | | | | | |
|---|---|---|---|---|---|---|---|---|
| Time of collection, | 13:00 | (3) | 13:00 | (3) | 13:00 | (2) | 13:00 | (3) |
| hrs (SD) | | | | | | | | |
| Time since last | 2.8 | (2.5) | 2.9 | (2.5) | 3.0 | (2.9) | 2.1 | (1.1)^{a} |
| meal, hrs (SD) | | | | | | | | |
| Dietary restrictions, | 50 | (13) | 28 | (15) | 10 | (8) | 12 | (18) |
| no. (%) | | | | | | | | |

Neuropsychiatric factors

| | | | | | | | | |
|---|---|---|---|---|---|---|---|---|
| Communication, | 83 | (23) | 73 | (20) | 103 | (17)^{a} | 79 | (18)^{a} |
| VABS-II standard | | | | | | | | |
| score (SD) | | | | | | | | |
| Socialization, | 85 | (23) | 73 | (15) | 108 | (18)^{a} | 82 | (20)^{a} |
| VABS-II standard | | | | | | | | |
| score (SD) | | | | | | | | |
| Activities of daily | 85 | (20) | 75 | (15) | 103 | (15) | 83 | (19)^{a} |
| living, VABS-II | | | | | | | | |
| standard score (SD) | | | | | | | | |

| | | | | | | |
|---|---|---|---|---|---|---|
| Social affect, | — | 13 | (5) | — | 5 | (3)^{a} |
| ADOS-II score (SD) | | | | | | |
| Restrictive/repetitive | — | 3 | (2) | — | 1 | (1)^{a} |
| behavior, ADOS-II | | | | | | |
| score (SD) | | | | | | |
| ADOS-II total score | — | 16 | (6) | — | 6 | (4)^{a} |
| (SD) | | | | | | |

In machine learning, using too many features in a training model can lead to overfitting. Overfitting is a case where once trained using training samples that include a large number of features, the machine learning model primarily only knows the training samples that it has been trained for. In other words, the machine learning model may have difficulty recognizing a sample that does not substantially match at least one of the training samples and it is therefore not general enough to identify variations of the feature set that are in fact associated with the target condition. It is desirable for a machine learning model to generalize to an extent that it can correctly recognize a new sample that differs from, but is similar-enough to, training samples to be associated with the target condition. On the other hand, it is also desirable for a machine learning model to include the most important features for accurately determining the presence or absence of the existence of a medical condition, ie those that differ the most between people with and without a target medical condition.

The present disclosure includes transformations of raw data to enable meaningful comparison of features, feature selection and ranking to create a Master Panel of ranked features with which the Test Model will be developed, and test model development that determines the fewest number of features that are necessary to achieve the highest performance accuracy and uses the features to implement a test model that defines a classification boundary that separates people with and without the target medical condition. The present disclosure includes testing that compares a test panel comprised of patient measures, human microtranscriptome, and microbial transcriptome features extracted from a patient's saliva against the implemented test model.

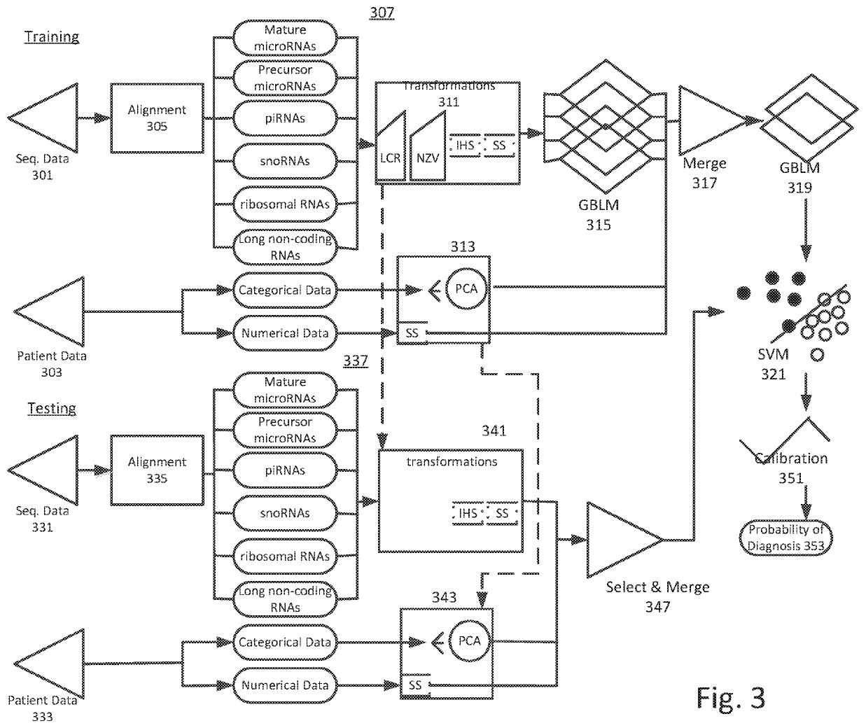

FIG. 3 is a system diagram for development and testing a machine learning model for diagnosing a medical condition in accordance with exemplary aspects of the disclosure. The machine learning methods that will be used for constructing the test model may be optimized by first transforming the raw data into normalized and scaled numeric features. Data may need to be corrected using standard batch effects methods, including within-lane corrections and between-lane corrections, and normalizing according to house-keeping RNAs. The data transformation methods used in the invention are chosen to facilitate identification of the RNA biomarkers with the most variability between the normal and target condition states and to convert, or transform, them to a unified scale so that disparate variables can meaningfully be compared. This ensures that only the most meaningful features will be subjected to analysis and eliminates data that could obscure or dilute the meaningful information.

The inputs required for application of the method may include the patient data described above and the relative quantities of the RNA biomarkers present in a saliva sample. Several methods of preparing biological samples containing extracellular RNA biomarkers and quantifying the relative amounts of RNA in the sample are known, and selection of a set of appropriate methods is a prerequisite to optimizing the inputs to be used for the method.

Transforming Data into Features

In 301, one or more processes to quantify RNA abundance in biological tissues may include the following: perform RNA purification to remove RNases, DNA, and other non-RNA molecules and contaminants; perform RNA quality assurance as determined by the RNA Integrity Number (RIN); perform RNA quantification to ensure sufficient amounts of RNA exist in the sample; perform RNA sequencing to create a digital FASTQ format file; perform RNA alignment to match sequences to known RNA molecules; and perform RNA quantification to determine the abundance of detected RNA molecules.

The RNA Integrity Number is a score of the quality of RNA in a sample, calculated based on quantification of ribosomal RNA compared with shorter RNA sequences, using a proprietary algorithm implemented by an Agilent Bioanalyzer system. A higher proportion of shorter RNA sequences may indicate that RNA degradation has occurred, and therefore that the sample contains low quality or otherwise unstable RNA.

RNA sequencing itself may include many individual processes, including adapter ligation, PCR reverse transcription and amplification, cDNA purification, library validation and normalization, cluster amplification, and sequencing.

Sequencing results may be stored in a single FASTQ file per sample. FASTQ files are an industry standard file format that encodes the nucleotide sequence and accuracy of each nucleotide. In the event that the sequencing system used generates multiple FASTQ files per sample (i.e., one per sample per flow lane), the files may be joined using conventional methods. The FASTQ format has four lines for each RNA read: a sequence identifier beginning with “@” (unique to each read, may optionally include additional information such as the sequencer instrument used and flow lane), the read sequence of nucleotides, either a line consisting of only a “+” or the sequence identifier repeated with the “@” replaced by a “+”, and the sequence quality score per nucleotide.

@SIM:1:FCX:1:15:6329:1045 1:N:0:2
TCGCACTCAACGCCCTGCATATGACAAGACAGAATC
+
<>;##=><9=AAAAAAAAAA9#:<#<;<<<????#=

The quality scores on the fourth line encode the accuracy of the corresponding nucleotide on the second line. A quality score of 30 represents base call accuracy of 99.9%, or a 1 in 1000 probability that the base call is incorrect. After sequencing a quality control step may be performed to ensure that the average read quality is greater than or equal to a threshold ranging from 28 to 34.

Optionally, other score encoding systems may be used, and other quality scores may be used. For example, the previously mentioned RIN may also be used as a quality assurance step, ideally with MN values greater than 3 passing quality assurance, or a quality control check requiring sufficient numbers of reads in the FASTQ (or comparable) file may be used.

Data may be directly uploaded from the sequencing instrument to cloud storage or otherwise stored on local or network digital storage.

In 305, alignment is the procedure by which sequences of nucleotides (e.g., reads in a FASTQ file) are matched to known nucleotide sequences (e.g., a library of miRNA. sequences, referred to as reference library or reference sequence). Sequencing data is processed according to standard alignment procedures. These may include trimming adapters, digital size selection, alignment to references indexes for each RNA category. Alignment parameters will vary by alignment tool and RNA category, as determined by one skilled in the art.

In 307 RNA features are categorized and at least one feature from each category is selected. RNA categories may include but are not limited to microRNAs (miRNAs; including precursor/hairpin and mature miRNAs), piwi-interacting RNAs (piRNAs), small interfering RNAs (siRNAs; also referred to as silencing RNAs), small nuclear RNAs (snRNAs), small nucleolar RNAs (snoRNAs), ribosomal RNAs (rRNAs), long non-coding RNAs (lneRNAs), microbial RNAs (coding &, non-coding), microbes identified by detected RNAs, the products regulated by the above RNAs, and the pathways in which the above RNAs are known to be involved. These categories may be further subdivided according to physical properties such as stage in processing (in the case of primary, precursor, and mature miRNAs) or functional properties such as pathways in which they are known to be involved.

Many aligning tools exist; sequence aligning is an area of active research. Although different aligners have different strengths and weaknesses, including tradeoffs for sequence length, speed, sensitivity, and specificity, aligners disclosed here may be replaced by a method with comparable results.

Skilled use of alignment tools is required to implement the method. Alignment parameters vary by alignment tool and RNA category, For example, parameters common to many sequence aligners include percent of match between read sequence and reference sequence, minimum length of match, and how to handle gaps in matches and mismatched nucleotides.

RNA alignment results in a BAM file which may then be quantified. BAM format is a binary format for storing sequence data. It is an indexed, compressed format that contains details about the aligned sequence reads, including but not limited to the nucleotide sequence, quality, and position relative to the alignment reference.

Quantification is the procedure by which aligned data in a BAM file is tabulated as number of reads that match a known sequence in a reference library. Individual reads may contain biologically relevant sequences of nucleotides that are mapped to biologically relevant molecules of non-coding RNA. RNA nucleotide sequence reads may be overlapping, contiguous, or non-contiguous in their mapping to a reference, and such overlapping and contiguous reads may each contribute one count to the same reference non coding RNA molecule.

Thus, nucleotide sequences read from a sequencing instrument (contained in FASTQ format), which are then mapped to a reference (BAM format), are then counted as matches to individual segments of the reference (i.e. RNAs), resulting in a list of nucleotide molecules and a count for each indicating the detected abundance in the biological sample.

Conversely, to detect the abundance of RNAs in a biological sample, the number of RN, reads that match each reference is tabulated from the aligned (BAM format) data.

The quantification method described above specifically works for human RNA reference libraries, and it may also work for microbial RNA reference libraries. An optional method for quantifying microbial RNA content includes the additional step of quantifying not only the reference sequences, but additionally the microbes from which the reference sequences are expressed.

Optionally, rather than quantifying the microbial RNA abundance, as described above using RNA-sequencing, quantification of the microbes themselves may be performed using 16S sequencing. 16S sequencing quantifies the 16S ribosomal DNA as unique identifiers for each microbe. 16S sequencing and the resultant data may be used instead of, or in conjunction with, microbial RNA abundance. For example, the 16S sequencing may be performed as a complement to confirm presence of microbes, wherein 165 confirms presence, and RNA-seq determines expression or abundance of RNAs, or cellular activity of the confirmed microbiota.

Optionally, after the identification of a panel of specific RNAs that are identified (in steps detailed below), implementation may instead use more targeted, less broad sequencing methods, including but not limited to qPCR. Doing so will allow for faster sequencing, and therefore faster result reporting and diagnosis.

After the above sequencing, alignment to reference, and RNA quantification, RNA data is now in the format of a count of human RNAs and microbes identified by RNAs, per RNA category for every subject.

Optionally, another quality control step may be implemented to confirm sufficient quantified RNA, in terms of either total alignments or the specific RNAs that are identified in the steps detailed below.

Corrections for batch effects may be required. Persons skilled in the art will recognize that methods to do so include modeling the RNA data with linear models including batch information, and subtracting out the effects of the batches.

The patient data also requires initial processing for use in the machine learning methods employed to develop the Test Model. In 303, patient data collected via questionnaire is preferably digitized, either through entry into spreadsheet software or digital survey collection methods. Optionally, steps may be taken to confirm data entry is correct and that all fields are complete, or missing data is imputed, or reject the subject or repeat data collection if data is suspected to be incorrect or is largely missing. Patient data is now in the format of numerical, yes/no, and natural language answers, per subject.

A randomly selected percent of data samples ranging from 50% to 10% may be set aside for testing purposes. This data is termed the “test data”, “test dataset”, or “test samples”. The data not included in the test dataset is termed the “training data”, “training dataset”, or “training samples”. The test dataset should not be inspected or visualized aside from previously mentioned quality control steps. Those skilled in the art will recognize that this method ensures that predictive models are not overfit to the available data, in order to improve generalizability of the models. Data transformation parameters, such as feature selection and scaling parameters, may be determined on the training data and then applied to both the training data and testing data.

Persons skilled in the art will recognize that statistical modeling and machine learning generally require data to be in specific formats that are conducive to analysis. This applies to both quantitative/numeric data and qualitative language-based information. Accordingly, in 313 non-numerical patient data are factorized, in which each feature or description is converted to a binary response. For example, a written description including a diagnosis of ADHD would become a 1 in an ‘has ADDH’ patient feature, and a 0 in the same category would represent a lack (or absence of reported) of ADHD diagnosis.

Factorization may lead to a large number of sparse and potentially non-informative or redundant categorical features, and to address this problem, dimensionality reduction may be used. Examples of dimensionality reduction include factor analysis, principal component analysis (PCA), linear discriminant analysis, and autoencoders. It may not be necessary to retain all dimensions, and a person skilled in the art may select cutoff thresholds visually or using common values or algorithms.

Many machine learning approaches display increased performance when input data are commensurate. Accordingly, patient data may be centered on zero (by removing the mean of each feature) and scaled. Scaling may be accomplished by dividing data by the standard. deviation or adjusting the range of the data to be between −1 and 1 or 0 and 1,

Additionally, many machine learning approaches display increased predictive performance on data drawn from normal distributions; Box-Cox or Yeo-Johnson transformations may be applied to adjust non-normal distributions.

Additionally, to ensure that outliers are commensurate with non-outliers and do not have undue influence, spatial sign (SS) transformation may be applied. This transformation is a group transformation in which data points are divided by group norm (SS(w)=w/∥w∥). The SS transformation may be applied either to all patient features collectively, or to subsets of patient features, or to some subsets of patient features and not others.

Optionally, other data transformations may be used in addition or as replacements. Further, data may not undergo transformation. A person skilled in the art may determine which transformations to use and when, and may rely on subsequent model performance in choosing between options.

Optionally, the above transformations and methods may be selected for different features or groups of features independently, rather than to all patient data indiscriminately.

Just as it is preferred to perform certain data transformations on patient data, RNA data may similarly benefit from selection of data, dimensionality reduction, and transformation. In 311, these steps may be applied to all RNA simultaneously, within RNA categories, or differently across RNA categories. In most cases, all biological data requires some data transformation to ensure that data values are commensurate, and to accommodate for variations in sequencing batches and other sources of variability.

As many of the RNAs comprising the oral transcriptome will have very low RNA counts, those with no counts or low counts may be removed. One method known to people skilled in the art is to only retain RNAs with more than X counts in Y % of training samples, where X ranges from 5 to 50, and ‘Y ranges from 10 to 90. Another method is to remove RNA features for which the sum of counts across samples are below a threshold of the total sum of all counts, or below a threshold of the total surer of the category of RNA counts to which the RNA belongs. This threshold may range from 0.5% to 5%.

Additionally, many of the RNA features may be largely stable across samples, regardless of the disease/disorder state of the patient from whom the sample was obtained. These features will show very low variance, and may be removed. The threshold of this variance may be set as a fixed number relative to the variance of other RNA features wherein the variance is from all RNAs or only those RNAs belonging to the same category as the RNA in question. In this case the threshold should be less than 50% but more than 10%. In an alternative method, within each RNA category features with a frequency ratio greater than A and fewer distinct values than B % of the number of samples, where the frequency ratio is between the first and second most prevalent unique values. A may range between 15 and 25, and B may range between 1 and 20. For example, in a population of 100 samples, if A is 19 and B is 10%, a feature with less than 10 unique values (less than frequency ratio of 19) and more than 95 of the sample contain the same value (less than 10%), the feature will be removed.

Additionally, RNA features described as above as showing low variance may instead be used as “house-keeping” RNAs to normalize other RNAs.

Optionally, a log or log-like transformation of count values may be performed. Many machine learning methods show improved predictive performance when input features have normal distributions. As RNA abundance levels often follow exponential distributions, the natural log, log₂ or log₁₀ may be taken of raw count values. To prevent count values of 0 becoming undefined, a small constant may be added to all samples. This value may range from 0.001 to 2, often 1. Another method, which eliminates the necessity of defining a constant, is to use a log-like transformation, such as inverse hyperbolic sine (IHS), defined as f(x)=In(x+\sqrt{x²+1}).

Optionally, as with patient data, RNA data may further benefit from spatial sign (SS) transformation. This group transformation may be applied collectively to all RNAs, or individual selectively within RNA categories. Spatial sign requires data to be centered first.

As discussed above, parameters, thresholds, and factors used to transform data are to be stored, saved, retained for use on test samples, such that test samples are transformed in an identical way to training samples.

Optionally, other data transformations may be used, either in replacement or conjunction with those described above. Some transformations may provide improved predictive power by being applied to multiple categories simultaneously. Different transformations, combinations of transformations, and parameterizations of transformations may be selected and applied for each RNA category independently.

Optionally, some categories of biomarkers and patient data may provide improved predictive power if they are first subdivided and transformed independently, as determined by expert knowledge, empirical predictive performance, or correlations with disease status.

Optionally, some or all of the above described transformations may be omitted.

These decisions may be made by one skilled in the art, as dependent on model performance in subsequently described steps.

In one embodiment, in 311, each category (e.g., piRNA) or subcategory (e g., mature miRNA) undergoes low count removal (LCR), near-zero variance (NZV) removal, inverse hyperbolic sine (HIS) transformation, and spatial sign (SS) group transformation. After these steps, biological data has been transformed into features, which will be prepared for further feature selection and ranking before being merged and handled jointly.

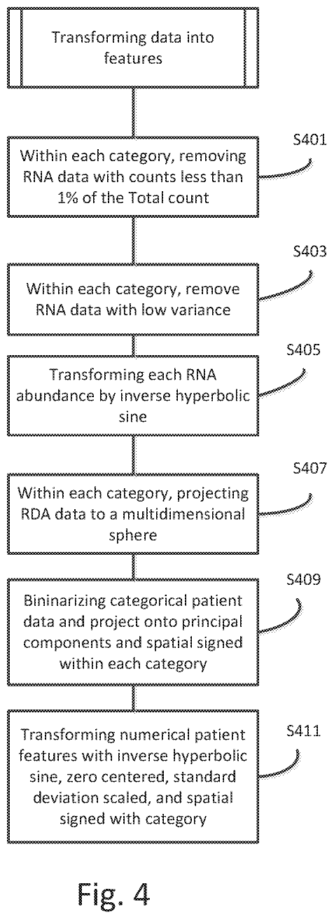

FIG. 4 is a flowchart for transforming data into features of FIG. 1. Data are transformed within categories, which consist of human microtranscriptome and microbial transcriptome type and categorical or numerical patient data. In S401, within each category, RNA features with counts less than 1% of the total counts are removed. In S403, within each category, features with low variance are eliminated. Such features have a frequency ratio greater than 19 and fewer distinct values than 10% of the number of samples, where the frequency ratio is between the first and second most prevalent unique values. In S405, each RNA abundance is centered on 0 and scaled by the standard deviation. Each RNA abundance is inverse hyperbolic sine transformed. In S407, within each RNA category, RNA features are projected to a multidimensional sphere using the spatial sign transformation. Spatial sign transformation additionally increases robustness to outliers.

In S409, categorical patient features are split into binary factors, where a 0 indicates absence, and 1 indicates presence of characteristic. Categorical patient features are then projected onto principal components that account for 80% of variance. In S411, numerical patient features are inverse hyperbolic sine transformed, zero centered, standard deviation scaled, and spatial signed within category.

### Feature Selection and Ranking

Different model input features may have different contributions or importance in predictive modeling, Further, some features may provide improved predictive performance when used in conjunction with others rather than alone. Accordingly, features are preferably ranked in importance, creating what may be referred to as a Variable Importance in Projection (VIP) score, or creating a list of features ranked in order of importance.

Statistical methods that consider individual features, like the Kruskal-Wallis test, PLSDA, and information gain, may be used to provide a VIP score, allowing ranking of input features. Kruskal-Wallis and similar statistical tests may be used to determine if different groups have different distributions of counts of RNAs, but investigate each feature independently. PLSDA is multivariate, and accordingly may be used to determine importance across multiple features in conjunction, but is limited to linear relations, both between features and between features and the disease/disorder state. Information gain compares the entropy of the system both with and without a given feature, and determines how much information or certainty is gained by including it.

Multivariate machine learning methods are not limited to linear relationships, and allow for interactions between features. Non-linear methods of analysis alloy for snore nuanced and precise relationships to be detected. Although machine learning models may have intrinsic methods to determine the importance of features, or even automate dropping features whose importance is negligible, in one embodiment a procedure to determine feature importance consists of comparing model performance both with and without a given feature. The comparison procedure provides an estimate of that feature's predictive power, and may be used to rank features in order of predictive power, or importance.

The choice of features can affect the accuracy of a prediction. Leaving out certain features can lead to a poor machine learning model. Similarly, including unnecessary features can lead to a poor machine learning model that results in too many incorrect predictions. Also, as mentioned above, using too many features may lead to overfitting. Ranking features in order of importance for a machine learning model and remove the least important features may increase performance,

Referring to FIG. 3, in 315, a random forest variant of a stochastic gradient boosting logistic regression machine (GBM) is used to rank the importance of features. GBMs are models in which ensembles of small, weak learners are aggregated, providing significant performance boosts over simpler methods.

GBMs utilize multivariate logistic regression in which the probability of a condition is a linear function of the input parameters subsequently fit to a logistic function: p(C)=1/1+exp(−α*X), where x is the weighted sum of features X=β₀+β₁x₁+β₂x₂+ . . . +βₙxₙ, from 1 to n. Each logistic regression machine is constrained by a maximum number of features and the number of samples it has access to in each iteration.

Random forests are known to learn training data very well, but as such are prone to overfitting the data and accordingly do not generalize well. Although gradient boosting machines may be used to predict a disease state, in this case they are used for selection and ranking of features to be used downstream. The goal of this stage is to create category-specific panels of RNAs that are maximally differentiated in the presence or absence of the target medical condition, and therefore maximally informative about the presence or absence of the condition.

In 315, each learner is a multivariate logistic regression model, comprised of 4-10 features((weak learning machines). Each iteration is built on a random subset of training samples (stochastic gradient boosting), and each node of the tree must have at least 20-40 samples. Model parameters include the number of trees (iterations) and size of the gradient steps (“shrinkage”) between iterations, Parameter values are selected by building multiple models, each with a unique combination of values drawn from a reasonable range, as known by those skilled in the art. The models are ranked by predictive performance (e.g., AUROC described below) across cross-validation resamples, and the parameter values from the best model are selected.

Characteristics and parameters specific to GBMs provide important benefits. The limited number of features reduces the possible overfitting of each tree, as does requiring a minimum number of observations. Further, cross-validation is used to reduce the likelihood that parameter values are selected from local minima. Models are fit using a majority of trials and performance is evaluated on the minority, and this process is repeated multiple times. For example, in 10-fold cross validation data is randomly split into 10ths (10 folds), each of which is used to test the performance of a model built on the other 9, giving 10 measures of performance of the model. In one embodiment, this process is repeated 10 times, giving 100 measures of performance of the model for the specific parameter values. This k-fold cross-validation is repeated j times to reduce the likelihood of overfitting (finding local minima) by training on a subset of data, and additionally provides more robust estimates of model performance.

Thus, the parameters controlling the number of trees and size of the gradient steps control the bias-variance trade off, improving performance while limiting over fitting. Further, the cross-validation is used to determine ideal parameters, and reduces over fitting.

Although each tree is a logistic regressor, and accordingly is a linear multivariate model whose output is fit to a logistic function, the combination of many such linear models allows for nonlinear classification.

To compare the predictive power of each input feature and thus determine a ranking, a model agnostic method is to compare the area under the receiver operator curve (AUROC) of models fit with and without the feature in question. The performance difference may be attributed to the feature, and the ranking of the value across features provides a ranking of the features themselves.

This ranking may be done within categories of RNAs, which also provides insight to the predictive power of each category of RNA. Alternatively, the ranking of features may be performed across categories, or subsets of categories, or groups of subsets of categories. Optionally, methods other than AUROC may be used for determining the variable importance of feature variables. A method for random forests is to count the number of trees in which a given feature is present, optionally giving higher weighting to earlier nodes. In some machine learning methods, the weighting coefficient may be used to rank features.

Optionally, methods other than GBMs or random forests may be used to rank features. Recursive feature elimination is an algorithm in which a model is trained with all features, the least informative feature is removed, the model is retrained, the next least informative feature is removed, and the process continues recursively. This algorithm allows for features to be ranked in order of importance, and may be used with any machine learning classifier, such as logistic regression or support vector machines, in the place of the feature ranking performed by GBMs.

Choice of features is an important part of machine learning construction. Analysis with a large number of features may require a large amount of memory and computation power, and may cause a machine learning model to be overfitted to training data and generalize poorly to new data. A gradient boosting machine method has been disclosed to rank input features. An alternative approach may be to use multiple different ranking methods in conjunction, and the results can then be aggregated (summed of weighted sum) to provide a single ranking. Other approaches to choosing an optimal set of features for a machine learning model also are available. For example, unsupervised learning neural networks have been used to discover features. As an example, self-organizing feature maps are an alternative to conventional feature extraction methods such as PCA. Self-organizing feature maps learn to perform nonlinear dimensionality reduction.

In some embodiments, machine learning feature ranking is applied to each RNA category independently, and the top RNA features from each is retained. The threshold for which features are retained may be determined empirically, and ideally the threshold may be set such that the number of features retained ranges from 5 to 50 % of the features for a given category. Note that the method for developing the Test Model can be performed using all features, rather than a select percent of features, but feature reduction reduces computational load. Additionally, all categories may be used, but low ranking in the subsequent master panel may drop some categories from remaining in the test panel.

After features are ranked within categories, a composite ranking model is built, using the top RNA features from each category and the patient data. This goal of this subsequent ranking model is to rank all features which will be used in the final predictive model. This composite ranking is referred to as the master panel 319.

The methods to compile the master panel may be similar to the methods used to compile the ranking for each RNA category, or may be drawn from options mentioned previously. Persons skilled in the art will recognize that different methods should, ideally, provide similar but not identical feature rankings. In some embodiments, the same method to determine category specific rankings is used to determine ranking in the master panel, for example GBM can be used for selecting and ranking both categorical features and the aggregate features across all categories which make up the master panel.

Optionally, within the master panel 319 the rank of individual features may be manually modified, based on expert knowledge of one skilled in the art. For example, RNAs known to vary with time of day (e.g., circadian miRNAs and microbes specific to certain geographic regions), BMI, age, or geographical region may be ranked highest to ensure that they are included in subsequent predictive models, thus accounting for variations in time of collection, weight, age, or region.

Alternatively, these RNAs or subsets of RNAs may be contraindicated and accordingly ranked lowest in the master panel, thus removing their influence, preventing the confounding influence of these variables. For example, sample saliva obtained too close to a time of last meal or time of last oral hygiene, including brushing teeth, mouth wash, may have a negative impact on a subset of the population of RNAs in the sample.

Thus, the master panel 319 is a list of features, ranked in order of importance or predictive power as determined both empirically with a machine learning model and by the judgment of one skilled in evaluating the target medical condition. Features may be grouped and ranked as a group, indicating that they have combined predictive power but are not necessarily predictive alone, or have reduced predictive power alone.

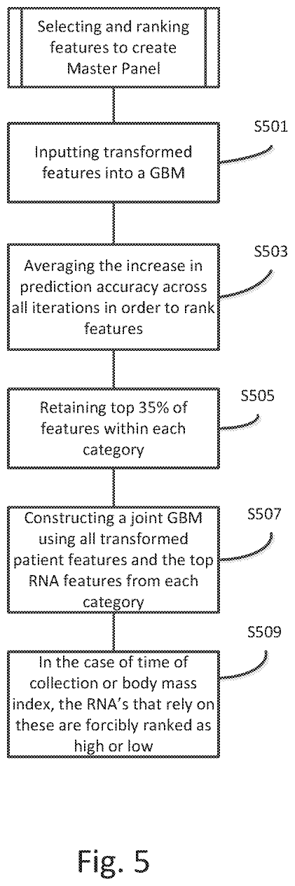

FIG. 5 is a flowchart for the feature selection and ranking step of an embodiment FIG. 1, In S501, the transformed human microtranscriptome and microbial transcriptome features are input to a stochastic gradient boosted logistic machine predictive model (GBM), where the outcome is 0 for non-disease state, and 1 for disease state. In S503, the increase in prediction accuracy for each feature is averaged across all iterations, allowing features to be ranked empirically. In S505, the top 35% of features within each category are retained.

In S507, a joint GBM model is constructed using all transformed patient features and the top performing RNA features from each transcriptome category. This model empirically ranks the features. In S509, in medical conditions in which predictions may be affected by patient features, such as time of collection (circadian variance) or BMI, the RNAs indicated for these conditions may be forcibly ranked as highest or lowest. Forcing the rank as high ensures that these RNA features will be retained in subsequent steps; forcing the rank to low ensures that these features will be eliminated in subsequent steps.

### Selecting a Test Panel of Features

In the next step of the method, a predictive test model is trained on the results of the feature ranking in the Master Panel. A test panel is the subset of features from the master panel which are used as input features in the predictive test model. In selecting the subset of features used for the test panel, features are usually (but not necessarily) considered in order of decreasing importance, such that the most important features are more likely to be included than less important features.

In some embodiments, the machine learning model that is used for feature selection and ranking (GBM) is different than the model chosen for selecting the reduced test panel and building the predictive model (e.g., support vector machine; SVM). The choice of different models for selection and ranking of features and for developing the Test Model and its test panel of features is made to benefit from the strengths of each machine learning model, while reducing their respective weaknesses. More specifically, it has been determined that random forest-type models learn training data very well, but potentially overfit, reducing generalizability. As such, random forest-based GBMs are used for feature selection and ranking, but not prediction. SVMs have been determined to have utility in biological count data and multiple types of data, and have tuning parameters that control overfitting, but are sensitive to noisy features in the data and accordingly may be less useful for feature selection.

Other machine learning algorithms that may be taught by supervised learning to perform classification include linear regression, logistic regression, naïve Bayes, linear discriminant analysis, decision trees, k-nearest neighbor algorithm, and neural networks. Support Vector Machines are found to be a good balance between accuracy and interpretability. Neural networks, on the other hand, are less decipherable and generally require large amounts of data to fit the myriad weights.

The machine learning method used to develop the Test Model and select the test panel from the master panel should be the same method used to later test novel samples once the diagnostic method is finalized. That is, if the predictive model to be applied to subjects is a support vector machine model, the method to select the test panel should be a similar or identical support vector machine model. In this way, the predictive performance of the test panel will be evaluated according to the way the test panel will be used.

The number of features in the test panel for the preferred predictive model may be determined by the fewest features that reach a plateau or approach an asymptote in predictive performance, such that increasing the number of features does not increase predictive performance in the training set, and indeed may degrade performance in the test set (overfitting).

In selecting and developing the test model, a grid of parameters may be used, wherein one axis is model class, another is model variants, number of features selected for training as another, and model parameters as another.

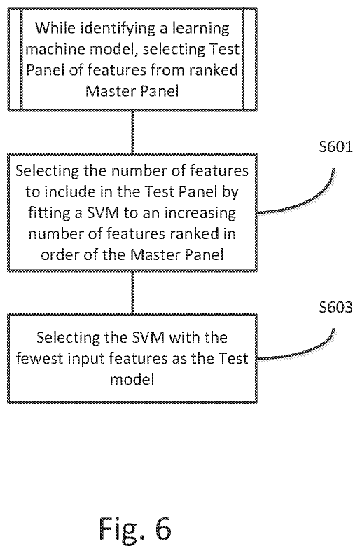

FIG. 6 is a flowchart for the method step in which a learning machine model and the associated test panel of features are developed. In S601, an SVM with radial kernel (321 in FIG. 3) is fit to an increasing number of features in ranked order from the Master Panel. When the predictive performance of the model reaches a plateau, the number of features provided as inputs for the round of training in which the plateau was achieved becomes the dimension of the Support Vector. The list of those features is the Test Panel. In S603, the SVM comprised of the set of Support Vectors with the fewest input features that has predictive performance on the plateau is selected as the Test Model.

A support vector machine is a classification model that tries to find the ideal border between two classes, within the dimensionality of the data. In the separable case, this border or hyperplane perfectly separates samples with a disorder/disease from those without. Although there may be an infinite number of borders which do so, the best border, or optimally separating hyperplane, is that which has the largest distance between itself and the nearest sample points. This distance is symmetrical around the optimally separating hyperplane, and defines the margin, which is the hyperplane along which the nearest samples sit. These nearest samples, which define both the margin and the optimal hyperplane, are called the support vectors because they are the multidimensional vectors that support the bounding hyperplane. Each support vector is an ordered arrangement of the features included in each training sample (xᵢ^{T}), and the list of those features is the test panel for that round of training.

To reduce overfitting on training data, a cost budget (C) is introduced, allowing some training samples to be incorrectly classified. In the non-separable case, in which no classifier may perfectly separate the training data into the correct classes, an error term (ϵ) is introduced. This allows training samples to be on the w g side of the margin, or on the wrong side of the hyperplane, and is called a “soft margin,”

The optimally separating hyperplane with a soft margin is defined by yᵢ(xᵢ^{T}β+β₀)≥1−ϵᵢ, ∀i for i . . . N samples, subject to ϵᵢ≥0 and Σᵢ₌₁^{N}ϵᵢ≤C, where y ∈ {−1,1} is the disease state status, xᵢ^{T} is a vector of the predictor inputs for sample i, β is a vector of the weights on the predictors, β₀ is the bias, and ϵᵢ is the error of sample i constrained by the cost budget.

The optimally separating hyperplane is that which has the largest margin surrounding the hyperplane, and is defined only by those xᵢ^{T} samples on the margin and on the incorrect side of the margin, which are the support vectors SV.

Calculating the optimally separating hyperplane is a quadratic optimization problem, and therefore can be solved efficiently. The goal is to maximize the margin (M) by finding optimal weights β and β₀ and ∥β∥=1, subject to the definition of the hyperplane yᵢ(xᵢ^{T}β+β₀)≥M(1−εᵢ) and restrictions on the error term (εᵢ≥0) and cost budget (Σᵢ₌₁ⁿεᵢ≤C). Note that εᵢ=0 for correctly classified training observations, εᵢ>0 for training observations on the incorrect side of the margin, and ϵᵢ >1 for incorrectly classified observations on the wrong side of the hyperplane.

An alternative definition of the optimally separating hyperplane allows for simplification and an efficient solution: the constraint ∥β∥=1 may be dropped by subjecting the optimization to

$$\frac{1}{‖ β ‖} {y}_{i} ( {x}_{i}^{T} β + {β}_{0} ) \geq M .$$

This formulation allows β and β₀ to be scaled by any constant or multiple, and lets

$$‖ β ‖ = \frac{1}{M} .$$

In this form, maximizing the margin is equivalent to minimizing ∥β∥. Further, minimizing ∥β∥ may be reformulated as minimizing ,1/2∥β∥², allowing among other things, the gradient to be linear and the optimization problem to be solved with quadratic programming.

Thus, the optimization problem is now defined as

$${min}_{β , {β}_{0}} \frac{1}{2} {‖ β ‖}^{2} + {CΣ}_{i = 1}^{N} {ɛ}_{i} ,$$

subject to yᵢ (xᵢ^{T}β+β₀)≥1−εᵢ, ∀i and εᵢ≥0. This is equivalent to the primal Lagrangian

$${ℒ}_{{P}_{β , {β}_{0}}} = \frac{1}{2} {‖ β ‖}^{2} + {CΣ}_{i - 1}^{N} {ɛ}_{i} - {Σ}_{i - 1}^{N} {α}_{i} [ {y}_{i} ( {x}_{i}^{T} β + {β}_{0} ) - ( 1 - {ɛ}_{i} ) ] - {Σ}_{i = 1}^{N} {μ}_{i} {ɛ}_{i} .$$

The dual problem (finding the minimum) is accordingly [symbol] _{D}=Σᵢ₌₁^{N}αᵢ−1/2Σᵢ₌₁^{N}Σⱼ₌₁^{N}αᵢαⱼyᵢyⱼxᵢ^{T}xⱼ. Note that αᵢ is the relative importance of each observation, such that αᵢ=>0 for support vectors and αᵢ=0 for non-support vectors, and thus i=1 . . . N may become i ∈ SV.

This convenient form makes clear an implementation of kernels, in which the dual problem may be written as [symbol] _{D}=Σ_{i ∈ SV}αᵢ−1/2Σ_{i ∈ SV}Σ_{j ∈ SV}αᵢαⱼyᵢyⱼ [symbol] h(xᵢ),h(xⱼ) [symbol] . As h(x) only requires the calculation of inner products, the specific transformation h(x) need not be provided, but may be replaced by a kernel function K(x,x′)= [symbol] h(x),h(x′) [symbol] .

A radial kernel, also known as a radial basis function or Gaussian, is defined by K(x,x′)=exp(−γ∥x−x′∥²), where λ is the radius or size of the Gaussian. Alternative kernel functions include polynomial kernels and neural network, hyperbolic tangent, or sigmoid kernels. A polynomial kernel of the dth-degree is defined by K(x,x′)=(1+ [symbol] x,x′ [symbol] ^{d}, where d is the degree of the polynomial. A neural network, hyperbolic tangent, or sigmoid kernel, is defined by K(x,x′)=tanh(k₁ [symbol] x,x′ [symbol] +k₂), where k₁ and k₂ define the slope and offset of the sigmoid.

SVM and kernel parameters are empirically derived, ideally with K-fold cross-validated training data in which 100/K % training samples are held out to measure the predictive performance, which may be repeated multiple times with different train/cross-validation splits. These parameters may be selected from a range expected to perform well, as known to persons skilled in the art, or specified explicitly.

If different kernels are used, relevant parameters may be derived as above.

Measures of predictive performance may include area under the receiver operator curve (AUC/AUROC/ROC AUC), sensitivity, specificity, accuracy, Cohen's kappa, F1, and Mathew's correlation coefficient (MCC).

The preferred number of features is found by building competing models with increasing numbers of input features, drawn in rank order from the master panel. Predictive performance, such as ROC or MCC, on the training data can then be viewed as a function of number of input features. The test model is the model with the fewest input features that approaches an asymptote or reaches a plateau of predictive performance. It is the model type with the best performance, with the kernel with the best performance, with the parameters with the best performance, requiring the fewest features.

The Test Model consists of the set of Support Vectors that were selected in the round of training that achieved maximum performance in classifying samples with the fewest features, and the dimension of the Support Vectors is equal to this smallest number of features. The list of features used in the samples for the round of training that yielded the Test Model set of Support Vectors is the Test Panel of features.

In one embodiment, the Support Vector Machine is used as the model class, with variant, radial kernel, features may range from 20 to 100; and model parameters include the cost budget (C) and kernel size (A).

### Analyzing Test Samples

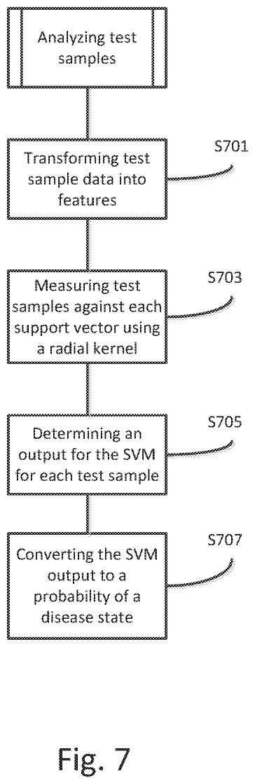

FIG. 7 is a flowchart for the test sample testing step of FIG. 1. Test samples represent a naïve sample from a subject or patient for whom the disease status is not known to the model, because the naïve sample was not used in training the test model. Test samples are new data on which the GBM and SVM models described above were not trained. Test samples are comprised of human microtranscriptome and microbial transcriptome and patient features that are included in the Test Panel; they need not include features which are removed prior to creating the Master Panel or not included in the Test Panel.

In S701, test sample features are transformed in the same way as the training samples were transformed, using parameters derived from the training data (FIG. 3, 331, 333, 335, 337, 341, 343, 347). These parameters include the mean for centering, standard deviation for scaling, and norm for spatial sign projection, as well as the trained SVM model (and also the fitted parametric sigmoid defined below for the Platt calibration).

As the optimally separating hyperplane is defined only by the support vectors, in S703 test samples need only be measured against each support vector in the Test Model, using the radial kernel defined above.

In S705, the output of the SVM Test Model, for test sample x*, is determined by a comparison of the sample against the set of Support Vectors comprising the Test Model. Specifically, the output is determined by f(x)=h(x)^{T}β+β₀=Σ_{i ∈ SV}αᵢyᵢK(xᵢ,x*), and is in the form of unsealed numeric values.

In some embodiments, the output of a Test Model includes class (disease status)and probability of membership to the class (probability of the disease). If the output is a value which does not explicitly indicate probability, the magnitude may be converted to a probability using a calibration method (FIG. 3, 351). The goal of such a method is to transform an unsealed output to a probability (FIG. 3, 353). Common calibration methods are the Platt calibration and isotonic regression calibration, although other methods are viable.

In the Platt calibration, the disorder/disease state and the magnitudes of the test model outputs are fit to a parametric sigmoid. The fitting parameters may be determined in the cross-validation folds mentioned previously for training the test model or derived in a separate cross-validation process. If the output of the trained SVM model for a test sample x is f(x)=Σ_{i ∈SV}αᵢyᵢK(xᵢ,x), then we may define the probability as P(y=1|f)=1/(1+exp(Af+B)), where P(y=1) is the probability of the disorder/disease state, and A and B are parameters to fit the sigmoid.

In S707, the SVM output is converted to a probability of disease state using Platt calibration, in which a parametric sigmoid is fit to cross-validated training data, and the assumption is made that the output of the SVM is proportional to the log odds of a positive (disease state) example. Thus,

$$P ( y = 1 \vert f ) = \frac{1}{1 + exp ( Af ( x ) + B )} .$$

Optionally, after definition of the Test Panel and parameters to create the Test Model, a Production Model may be built on both the training and testing dataset using the parameters from the Test Model. If this step is not performed, the Test Model may constitute the Production Model.

### Alternative Machine Learning Models

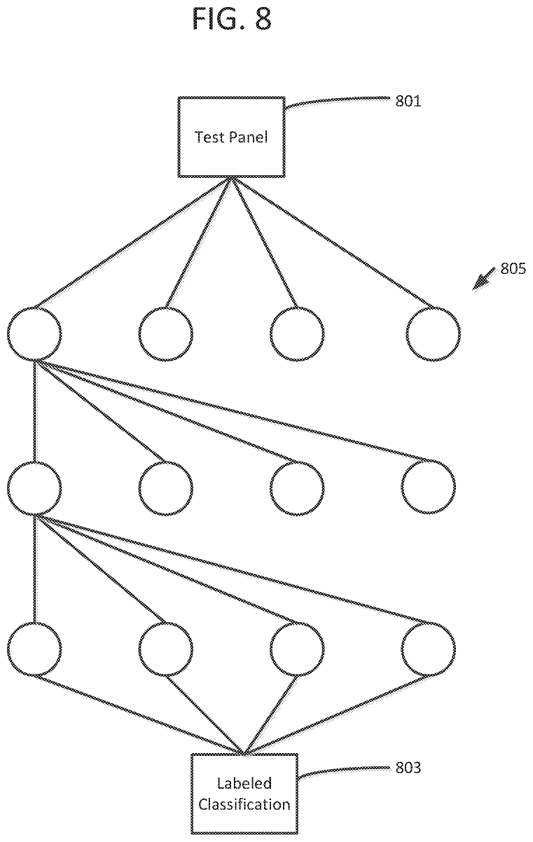

As the amount of data available for training a machine learning model increases, in particular related to diagnosis of mental disorders/diseases s as ASD and Parkinson's Disease, other machine learning methods may be used instead of, or in conjunction with, Support Vector Machines. FIG. 8 is a diagram for a neural network architecture in accordance with an exemplary aspect of the disclosure. The diagram shows a few connections, but for purposes of simplicity in understanding does not show every connection that may be included in a network. The network architecture of FIG. 8 preferably includes a connection between each node in a layer and each node in a following layer. Regarding FIG. 8, a neural network architecture may be provided with a panel of features 801 just as the Support Vector Machine of the present disclosure. The same output for classification 803 that was used for the Support Vector Machine model may also be used in the architecture of a neural network. Instead of learning a set of support vectors that define a classification boundary, a neural network learns weighted connections between nodes 805 in the network. Weighted connections in a neural network may be calculated using various algorithms. One technique that has proven successful for training neural networks having hidden layers is the backpropagation method. The backpropagation method iteratively updates weighted connections between nodes until the error reaches a predetermined minimum. The name backpropagation is due to a step in which outputs are propagated back through the network. The back propagation step calculates the gradient of the error. Also, similar to the support vector machine of the present disclosure, a neural network architecture may be trained using radial basis functions as activation functions.

Further, there are training methods for neural networks, as well as support vector machines, that enable them to be incrementally trained as more data becomes available. Incremental learning is a model in which a learning model can continue to learn as new data becomes available, without having to relearn based on the original data and new data. Of course, most learning models, such as neural networks, may be retrained using all data that is available.

Still further, the number of internal layers of a neural network may be increased to accommodate deep learning as the amount of data and processing approaches levels where deep learning may provide improvements in diagnosis. Several machine learning methods have been developed for deep learning. Similar to Support Vector Machines, deep learning may be used to determine features used for classification during the training process. In the case of deep learning, the number of hidden layers and nodes in each layer may be adjusted in order to accommodate a hierarchy of features. Alternatively, several deep learning models may be trained, each having a different number of hidden layers and different numbers of hidden nodes that reflect variations in feature sets.

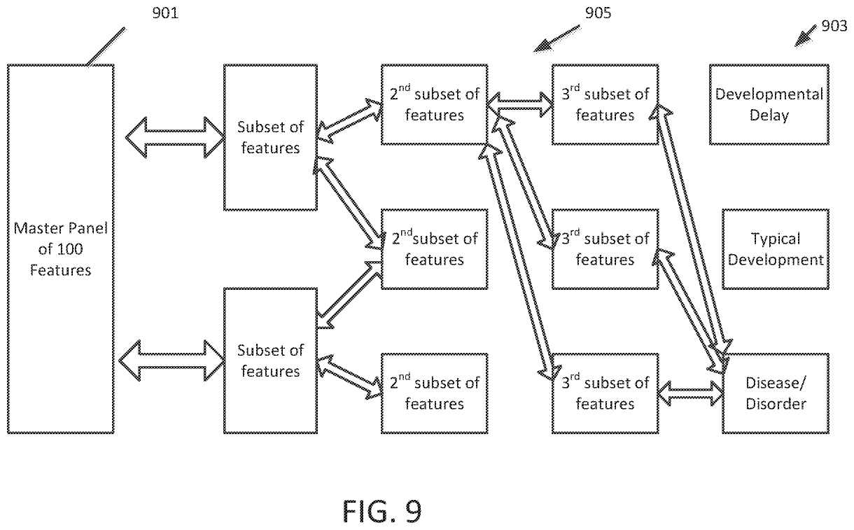

In some embodiments, a deep learning neural network may accommodate a full set of features froth a Master Panel and the arrangement of hidden nodes may themselves learn a subset of features while performing classification. FIG. 9 is a schematic for an exemplary deep learning architecture. As in FIG. 8, not all connections are shown. In some embodiments, less than fully interconnection between each node in the network may be used in a learning model. However, in most cases, each node in a layer is connected to each node in a following layer in the network. It is possible that some connections may have a weight with a value of zero. In addition, the blocks shown in the figure may correspond to one or more nodes. The input layer 901 may consist of a Master Panel of 100 features. In some embodiments, each feature may be associated with a single node. The series of hidden layers may extract increasingly abstract features 905, leading to the final classification categories 903.

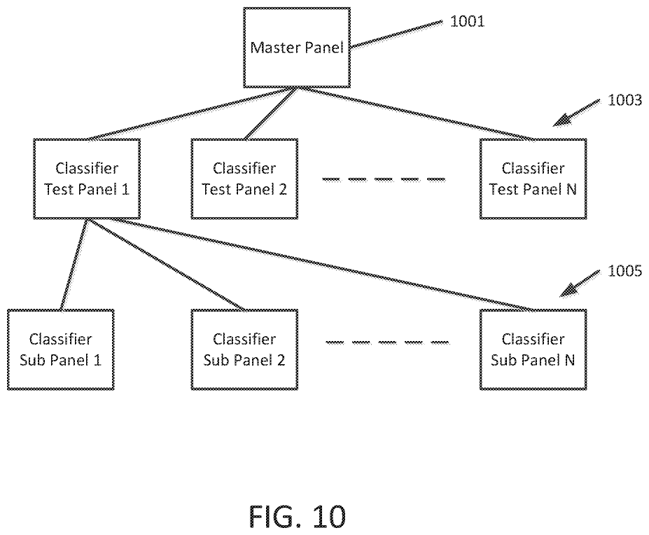

Deep learning classifiers may be arranged as a hierarchy of classifiers, where top level classifiers perform general classifications and lower level classifiers perform more specific classifications. FIG. 10 is a schematic for a hierarchical classifier in accordance with an exemplary aspect of the disclosure. Lower level classifiers may be trained based on specific features or a greater number of features. Regarding FIG. 10, one or more deep learning classifiers 1003 may be trained on a small set of features from a Master Panel 1001 and detect early on that a patient is clearly typical development, or clearly has a target disorder. bower level deep learning classifiers 1005 may have a greater number of hidden layers than higher level classifiers, and may consider a greater number of features in order to more finely discern the presence or absence of the target disorder in a patient.

### Example Machine Learning Model—ASD Diagnostics

There is a need to establish reliable diagnostic criteria for ASD as early as possible and, at the same time, differentiate those subgroups with distinct developmental concerns. However, a panel of biomarkers that has sufficient sensitivity and specificity must be identified in order to develop a useful molecular diagnostic tool for ASD. Defining the oral transcriptome profile and machine learning predictive model focused on the time of initial ASD diagnosis will help differentiate between ASD and non-ASD children, including those with DD.

In one embodiment, a machine learning model is determined as a diagnostic tool in detecting autism spectrum disorder (ASD). Multifactorial genetic and environmental risk factors have been identified in ASD. Subsequently, one or more epigenetic mechanisms play a role in ASD pathogenesis. Among these potential mechanisms are non-coding RNA, including micro RNAs (miRNAs), piRNAs, small interfering RNAs (siRNAs), small nuclear RNAs (snRNAs), small nucleolar RNAs (snoRNAs), ribosomal RNAs (rRNAs), and long non-coding RNAs (lncRNAs).

MicroRNAs are non-coding nucleic acids that can regulate expression of entire gene networks by repressing the transcription of mRNA into proteins, or by promoting the degradation of target mRNAs. MiRNAs are known to be essential for normal brain development and function.

miRNA isolation from biological samples such as saliva and their analysis may be performed by methods known in the art, including the methods described by Yoshizawa, et al., Salivary MicroRNAs and Oral Cancer Detection, Methods Mol Biol. 2013; 936: 313-324; doi: 10.1007/978-1-62703-083-0 (incorporated by reference) or by using commercially available kits, such as mirVana™ miRNA Isolation Kit which is incorporated by reference to the literature available at https://_tools.thermofisher.com/content/sfs/manuals/fm_1560.pdf (last accessed Jan. 9, 2018).

miRNAs can be packaged within exosomes and other lipophilic carriers as a means of extracellular signaling. This feature allows non-invasive measurement of miRNA levels in extracellular biofluids such as saliva, and renders them attractive biomarker candidates for disorders of the central nervous system (CNS). In fact, a pilot study of 24 children with ASD demonstrated that salivary miRNAs are altered in ASD and broadly correlate with miRNAs reported to be altered in the brain of children with ASD. A procedure has been developed to establish a diagnostic panel of salivary miRNAs for prospective validation. Using this procedure, characterization of salivary miRNA concentrations in children with ASD, non-autistic developmental delay (DD), and typical development (TD) may identify panels of miRNAs for screening (ASD vs. TD) and diagnostic (ASD vs. DD) potential.

miRNAs that may be good biomarkers for ASD include hsa-mir-146a, hsa-mir-146b, hsa-miR-92a-3p, hsa-miR-106-5p, hsa-miR-3916, hsa-mir-10a, hsa-miR-378a-3p, hsa-miR-125a-5p, hsa-miR146b-5p, hsa-miR-361-5p, hsa-mir-410, hsa-mir-4461, hsa-miR-15a-5p, hsa-miR-6763-3p, hsa-miR-196a-5p, hsa-miR-4668-5p, hsa-miR-378d, hsa-miR-142-3p, hsa-mir-30c-1, hsa-mir-101-2, hsa-mir-151a, hsa-miR-125b-2-3p, hsa-mir-148a-5p, hsa-mir-548I, hsa-miR-98-5p, hsa-miR-8065, hsa-mir-378d-1, hsa-let-7f-1, hsa-let-7d-3p, hsa-let-7a-2, hsa-let-7f-2, hsa-let-7f-5p, hsa-mir-106a, hsa-mir-107, hsa-miR-10b-5p, hsa-miR-1244, hsa-miR-125a-5p, hsa-mir-1268a, hsa-miR-146a-5p, hsa-mir-155, hsa-mir-18a, hsa-mir-195, hsa-mir-199a-1, hsa-mir-19a, hsa-miR-218-5p, hsa-mir-29a, hsa-miR-29b-3p, hsa-miR-29c-3p, hsa-miR-3135b, hsa-mir-3182, hsa-mir-3665, hsa-mir-374a, hsa-mir-421, hsa-mir-4284, hsa-miR-4436b-3p, hsa-miR-4698, hsa-mir-4763, hsa-mir-4798, hsa-mir-502, hsa-miR-515-5p, hsa-mir-5572, hsa-miR-6724-5p, hsa-mir-6739, hsa-miR-6748-3p, hsa-miR-6%70-5p, hsa_let_7d_5p, hsa_let_7e_5p, hsa_let 7g_5p, hsa_miR_101_3p, hsa_miR_1307_5p. hsa_miR_142_5p, hsa_miR_148a_5p, hsa_miR_151a_3p, hsa_miR 210_3p hsa_miR_28_3p, hsa_miR29a_3p, hsa_miR_3074_5p, hsa_miR_374a_5p.

Other non-coding RNAs, such as piRNAs, have been shown to also be good biomarkers for ASD. piRNA biomarkers for ASD include piR-hsa-15023, piR-hsa-27400, piR-hsa-9491, piR-hsa-29114, piR-hsa-6463, piR-hsa-24085, piR-hsa-12423, piR-hsa-24684, piR-hsa-3405, piR-hsa-324, piR-hsa-18905, piR-hsa-23248, piR-hsa-28223, piR-hsa-28400, piR-hsa-1177, piR-hsa-26592, piR-hsa-11361, piR-hsa-26131, piR-hsa-27133, piR-hsa-27134, piR-hsa-27282, piR-hsa-27728, wiRNA-1433, wiRNA-2533, wiRNA-3499, wiRNA-9843.

Ribosomal RNA that may be good biomarkers for ASD include RNA5S, MTRNR2L4, MTRNR2L8.

snoRNA that may be good biomarkers for ASD include SNORD118, SNORD29, SNORD53B, SNORD68, SNORD20, SNORD41, SNORD30, SNORD34, SNORD110, SNORD28, SNORD45B, SNORD92.

Long non-coding RNA that may be a good biomarker for ASS includes LOC730338.

In addition to panels, associations of salivary miRNA expression and clinical/demographic characteristics may also be considered. For example, time of saliva collection may affect miRNA expression. Some miRNA, such as miR-23b-3p, may be associated with time since last meal.

However, factors that may influence salivary RNA expression may also be crucial. For example, it is known that components of the oral microbiome may correlate with the diagnosis of ASD and/or specific behavioral symptoms. Microbial genetic sequence (mBIOME) present in the saliva sample that may be biomarkers for ASD include: Streptococcus gallolyticus subsp. gallolyticus DSM 16831, Yarrowia lipolytica CLIB122, Clostridiales, Oenococcus oeni PSU-1, Fusarium, Alphaproteobacteria, Lactobacillus fermentum, Corynebacterium uterequi, Ottowia sp. oral taxon 894, Pasteurella multocida subsp. multocida OH4807, Leadbetterella byssophila DSM 17132, Staphylococcus, Rothia, Cryptococcus gattii WM276, Neisseriaceae, Rothia dentocariosa ATCC 17931, Chryseobacterium sp. MB B 17019, Streptococcus agalactiae CNCTC 10/84, Streptococcus pneumoniae SPINA45, Tsukamurella paurometabola DSM 20162, Streptococcus mutans UA159-FR, Actinomyces oris, Comamonadaceae, Streptococcus halotolerans, Flavobacterium columnare, Streptomyces griseochromogenes, Neisseria, Porphyromonas, Streptococcus salivarius CCHSS3, Megasphaera elsdenii DSM 20460, Pasteurellaceae, and an unclassified Burkholderiales. Other microbes that may be biomarkers for ASD include Prevotella timonensis, Streptococcus vestibularis, Enterococcus faecalis, Acetomicrobium hydrogeniformans, Streptococcus sp. HMSC073D05, Rothia dentocariosa, Prevotella marshii, Prevotells sp. HMSC073D09, Propionibacterium acnes, Campylobacter, Arthrobacter, Dickeya, Jeatgalibacillus, Leuconostoc, Maribacter, Methylophilus, Mycobacteriutn, Ottowia, Trichormus. Further, other microbes that may be biomarkers for ASD include Actinomyces meyeri, Actinomyces radicidentis, Eubacterium, Kocuria flava, Kocuria rhizophila, Kocuria turfanensis, Lactobacillus fermentum, Lysinibacillus sphaericus, Micrococcus luteus, Streptococcus dysgalactiae.

Microbial taxonomic classification is imperfect, particularly from RNA sequencing data. Most, if not all, classifiers assign reads to the lowest common taxonomic ancestor, which in many cases is not at the same level of specificity as other reads. For example, some reads may be classified down to the sub-species level, whereas others are only classified at the genus level. Accordingly, some embodiments prefer to view the data only at specific levels, either species, genus, or family, to remove such biases in the data.

Another method to avoid such inconsistent biases are to instead interrogate the functional activity of the genes identified, either in isolation from or in conjunction with the taxonomic classification of the reads. As mentioned above, the KEGG Orthology database contains orthologs for molecular functions that may serve as biomarkers. In particular, molecular functions in the KEGG Orthology database that may be good biomarkers include K00088, K00133, K00520, K00549, K00963, K01372, K01591, K01624, K01835, K01867, K19972, K02005, K02111, K2795, K02879, K02919, K02967, K03040, K03100, K03111, K14220, K14221, K14225, K14232, K19972.

As mentioned above, a problem that affects use of biomarkers as diagnostic aids is that while the relative quantities of a biomarker or a set of biomarkers may differ in biologic samples between people with and without a medical condition, tests that are based on differences in quantity often are not sensitive and specific enough to be effectively used for diagnosis. An objective is to develop and implement a test model that can be used to evaluate the patterns of quantities of a number of RNA biomarkers that are present in biologic samples in order to accurately determine the probability that the patient has a particular medical condition.

An embodiment of the machine learning algorithm has been developed as a test model that may be used as a diagnostic aid in detecting autism spectrum disorder (ASD). In one embodiment, the test model is a support vector machine with radial basis function kernel. The number of features in the Test Panel found to achieve the asymptote of the predictive performance curve is 40. However, the number of features in a Test Panel is not limited to 40. The number of features in a Test Panel may vary as more data becomes available for use in constructing the test model.

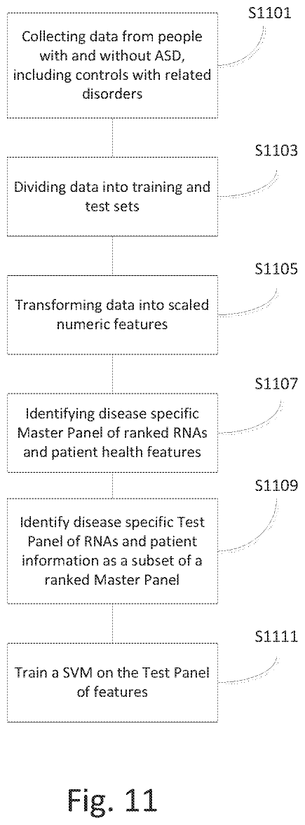

FIG. 11 is a flowchart for developing a machine learning model for ASD in accordance with exemplary aspects of the disclosure. In S1101, input data is collected from cohorts both with and without ASD, including controls with related disorders which complicate other diagnostic methods, such as developmental delays. In S1103, the data is split into training and test sets. In S1105, data is transformed using parameters derived on training data, as in 311 of FIG. 3.

Within each RNA category, abundance levels are normalized, scaled, transformed and ranked. Patient data are scaled and transformed. Oral transcriptome and patient data are merged and ranked to create the Master Panel.

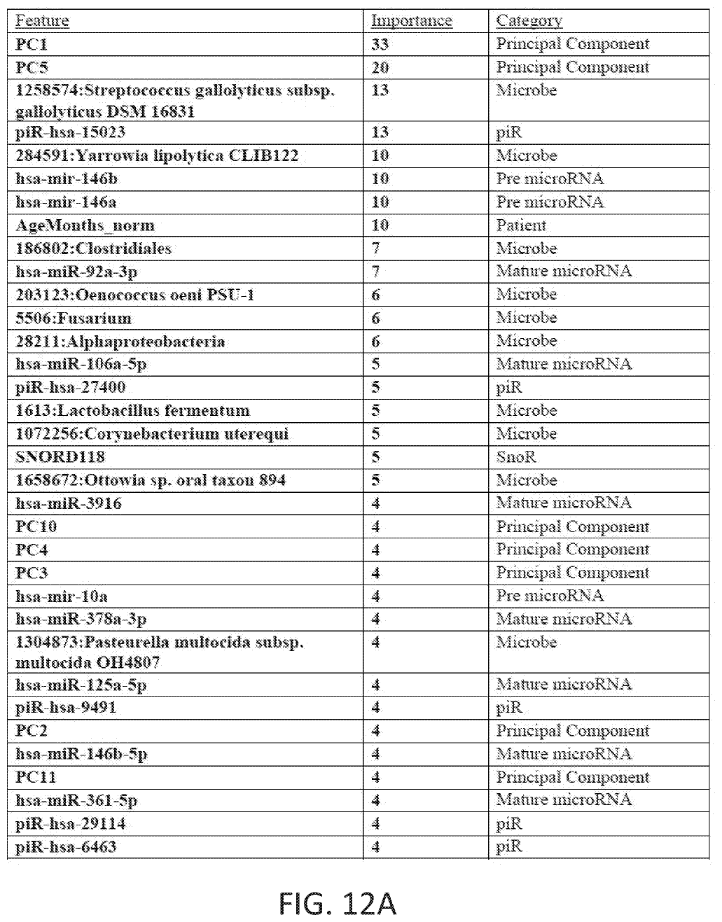

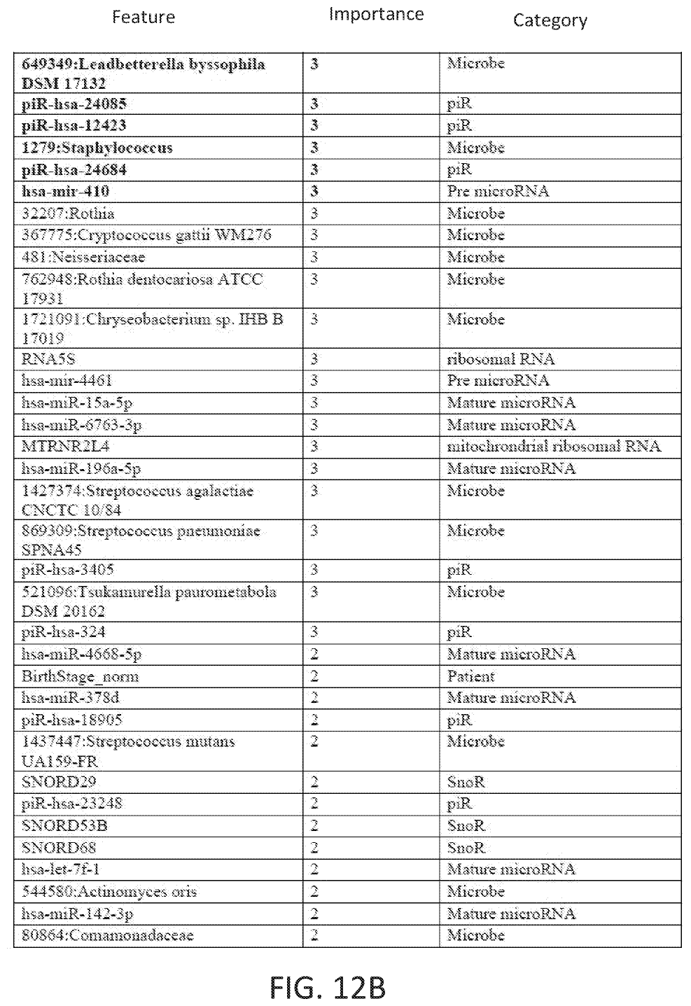

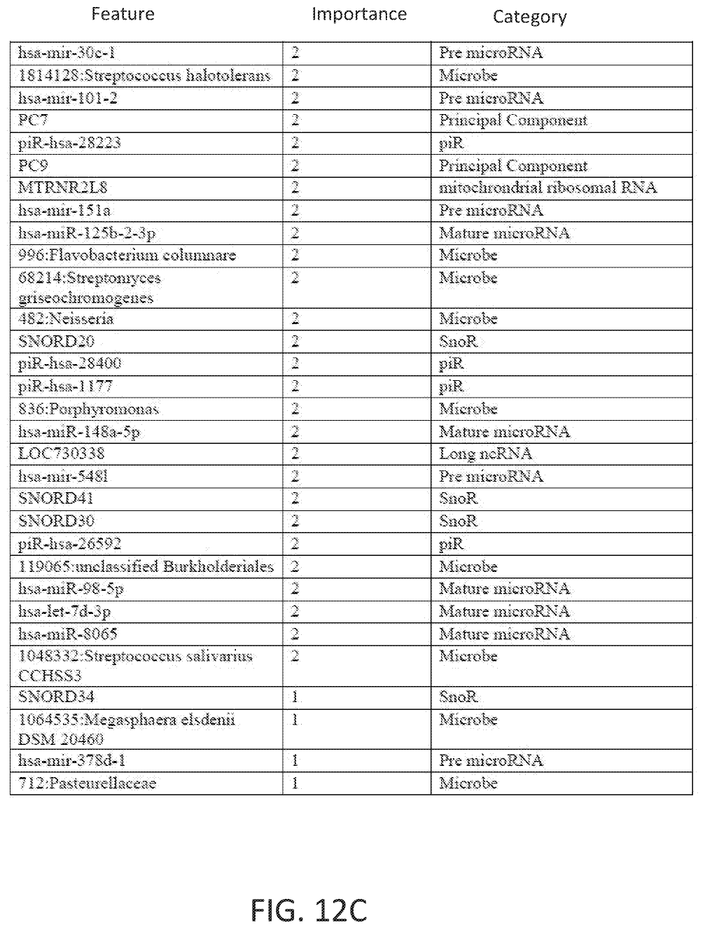

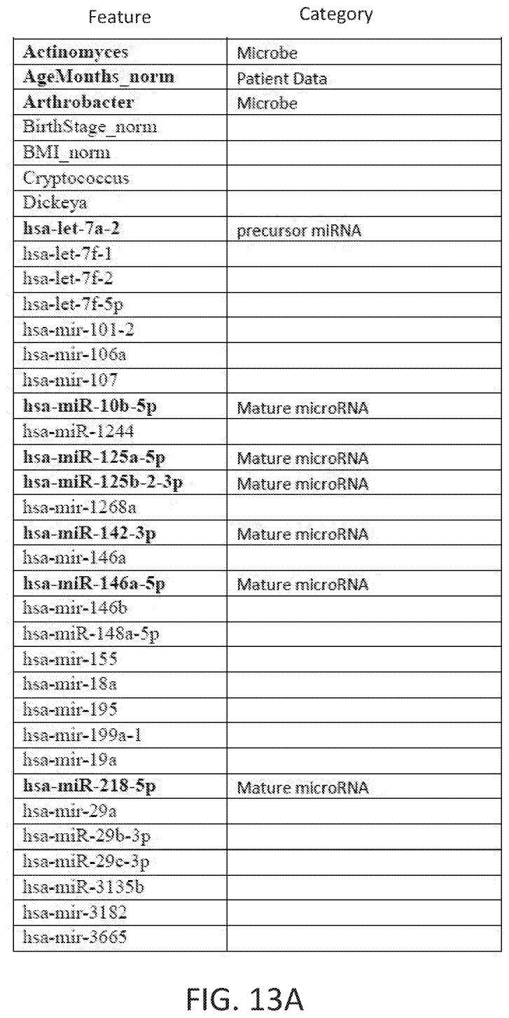

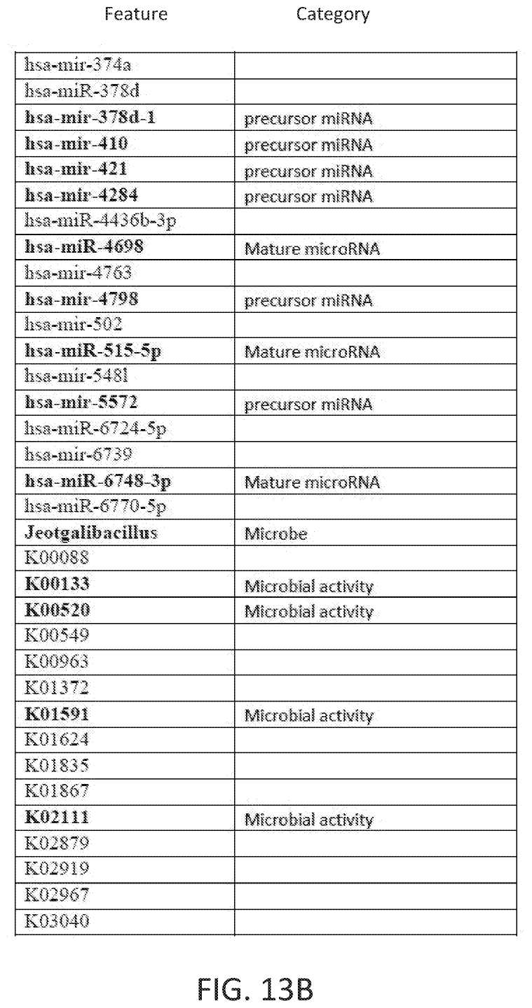

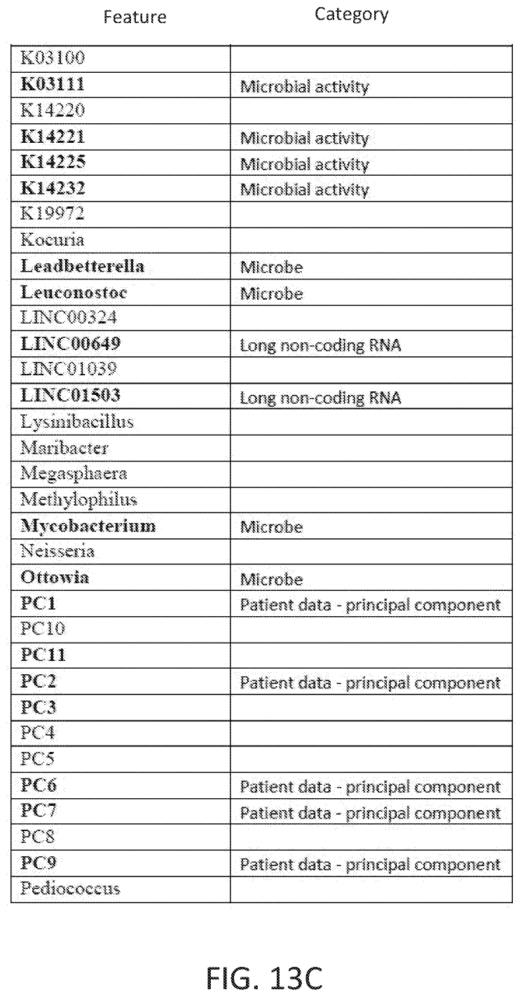

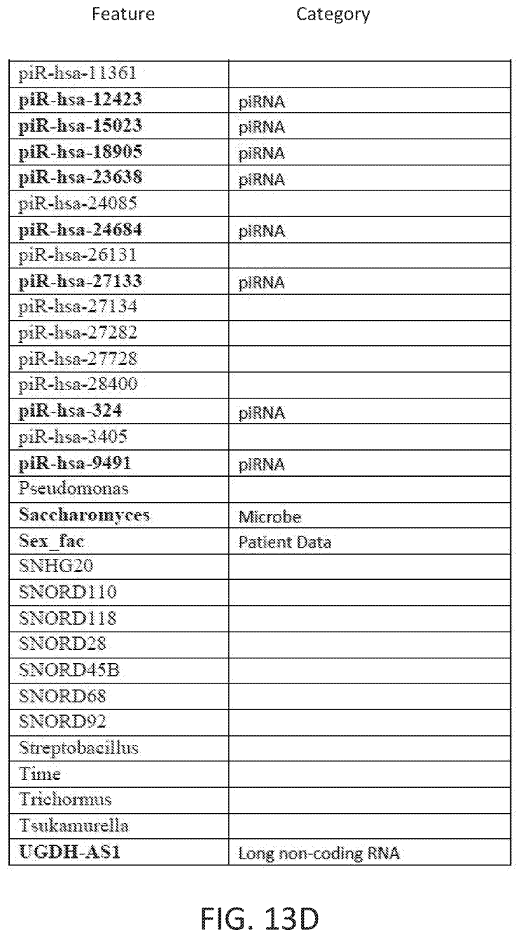

In S1107, a disease specific Master Panel of ranked RNAs and patient information is identified from which the Test Panel will be derived. The Master Panel is determined using the GBM model as in 315 of FIG. 3. FIGS. 12A, 12B and 12C are an exemplary Master Panel of features that has been determined based on the Meta transcriptome and patient history data for ASD The first column in the figure is a list of principal components, RNA, microbes and patient history data provided as the features. Features listed in the first column as PC1, PC2, etc. are principal components that are results of performing principal component analysis. The second column in the figure is a list of importance values for the respective features. The third column in the figure is a list of categories of the respective features. The number of features in the Master Panel is not limited to those shown in FIGS. 12A, 12B, 12C, because the features that make up the Master Panel may vary as the Test Model algorithm is updated to include in the development process more data or other methods. For example, FIGS. 13A, 13B, 13C, 13D are a further exemplary Master Panel of features that have been determined based on the Metatranscriptome and patient history data for ASD.

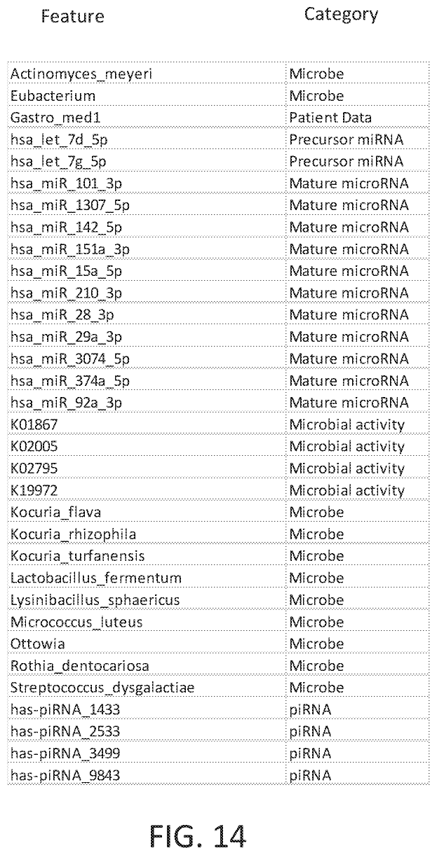

In S1109, a set of Support Vectors with elements consisting of a disease specific Test Panel of patient information and oral transcriptome RNAs is identified to be used for the Test Model. The Test Panel is a subset of a ranked Master Panel. Regarding FIGS. 12A, 12B and 12C, an exemplary Test Panel is the top 40 features listed in the Master Panel. Similarly, FIGS. 13A, 13B, 13C and 13D show, in bold, features that may be included in a Test Panel. FIG. 14 is an exemplary Test Panel of features that have been determined based on the Metatranscriptome and patient history data for ASD. The number of features may vary depending on the training data and the number of features that are required to reach a plateau in the predictive performance curve. The Test Panel may be derived from the Master Panel using the radial kernel SVM model as in 321. The SVM is trained in successive training rounds using increasing numbers of features in the Master Panel as inputs, until predictive performance levels off, i.e., reaches a plateau.

It has been determined that Test Panels derived using the SVM differ from the Test Panels of diagnostic microRNAs produced using methods without machine learning. Non-machine learning methods diagnosis a disease/condition by a generic comparison of abundances between test samples from normal subjects and subjects affected by the condition. The SVM derived Test Panels provide superior accuracy over the simple comparison of abundances of the non-machine learning methods.

In S1111, a Support Vector Machine Model is trained on increasing numbers of the features from the Master Panel of features. The Model determines an optimally separating hyperplane with a soft margin. This margin is defined by the support vectors, as described above. The Test Model is the support vector machine model with the fewest input parameters with comparable performance to SVMs with successively more input parameters. The Test Panel is the set of features that comprise the components of the support vectors used in the Test Model.

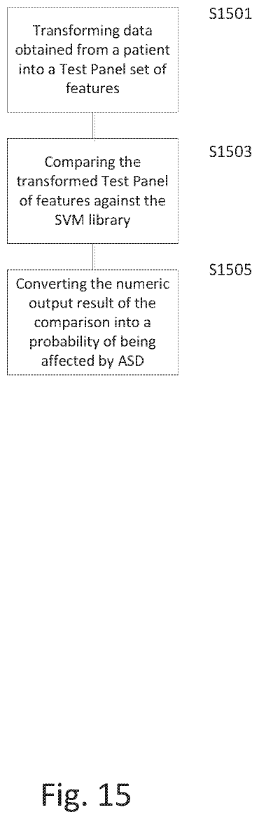

FIG. 15 is a flowchart for a machine learning model for determining the probability that a patient may be affected by ASD. In S1501, the Test Panel set of rave data (RNA abundances and patient information) obtained from the patient to be tested (RNA from saliva, patient information from interview) is transformed into a Test Panel set of Features as in 341 and 343 of FIG. 3. In S1503, the Transformed. Test Panel set of Features obtained from the patient is compared against the set of Support Vectors that define the classification hyperplane boundary (Support Vector Library), 321 in FIG. 3. Comparison of the Test Panel set of Features from the patient to be tested is compared against the Test Model's Support Vector Library using the comparison function f(x)=h(x)^{T}β+α₀=Σ_{i ∈ SV}αᵢyᵢK(xᵢ,x*). The output of the comparison is an unsealed numeric value.

In S1505, the numeric output result of the comparison of the Test Panel set of Features from the patient against the Test Model is converted into a probability of being affected by the ASD target condition using the Platt calibration method, as in 351 of FIG. 3.

The disclosed machine learning algorithms may be implemented as hardware, firmware, or in software. A software pipeline of steps may be implemented such that the speed and reliability of interrogating new samples may be increased. Accordingly, the required input data, collected from patients via questionnaire and sequenced saliva swab, are preferably processed and digitized. The biological data is preferably aligned to reference libraries and quantified to provide the abundance levels of biomarker molecules. These, and the patient data, are transformed as determined in the above steps, using parameters determined on the training data.

The data used for training the test model may be combined with data that had been used for determining a master panel in order to obtain a more comprehensive training set of data which may yield a Test Model and Test Panel that has better sensitivity and specificity in predicting the ASD target condition. The combined transformed data may then be used to develop the Production Model, the output of which is transformed using the calibration method, and a probability of condition is determined. Thus, the Production Model uses the same inputs and parameters as derived in the Test Model, but it is trained on both the training and test data sets. In this preferred embodiment, a Production Model to aid diagnosis of ASD is defined using a larger data set and a software pipeline is implemented. Biological samples have the RNA purified, sequenced, aligned, and quantified; patient data is digitized.

Subjects to be tested may have samples collected in the same manner as samples were collected from training subjects. Data from subjects to be tested preferably undergo identical sequencing, preprocessing, and transformations as training data. If the same methods are no longer available or possible, new methods may be substituted if they produce substantially equivalent results or data may be normalized, scaled, or transformed to substantially equivalent results.

Quantified features from test samples may at least include the test panel, but may include the master panel or all input features. Test samples may be processed individually, or as a batch.

A Test Panel is selected from the data, and data from both sources are transformed, likely using combinations of PCA, IHS, and SS. Transformed data are input into the Production Model, an SVM with radial kernel, and the output is calibrated to a probability that the patient has or does not have a medical condition, particularly, a mental disorder such as ASD or PD, a mental condition or a brain injury.

### Exemplary Application of the Disclosed Process

In a non-limiting example of application of the disclosed process, saliva is collected in a kit, for example, provided by DNA Genotek. A swab is used to absorb saliva from under the tongue and pooled in the cheek cavities and is then suspended in RNA stabilizer. The kit has a shelf life of 2 years, and the stabilized saliva is stable at room temperature for 60 days after collection. Samples may be shipped without ice or insulation. Upon receipt at a molecular sequencing lab, samples are incubated to stabilize the RNA until a hatch of 48 samples has accumulated.

At this time, RNA is extracted using standard Qiazol (Qiagen) procedures, and cDNA libraries are built using Illumina Small RNA reagents and protocols. RNA sequencing is performed on, for example, Illumina NextSeq equipment, which produces BCL files. These image files capture the brightness and wavelength (color) of each putative nucleotide in each RNA sequence. Software, for example Illumina's bcl2fastq, converts the BCL files into FASTQ files. FASTQs are digital records of each detected RNA sequence and the quality of each nucleotide based on the brightness and wavelength of each nucleotide. Average quality scores (or quality by nucleotide position) may be calculated and used as a quality control metric.

Third-party aligners are used to align these nucleotide sequences within the FASTQ files to published reference databases, which identifies the known RNA sequences in the saliva sample. An aligner, for example the Bowtie1 aligner, is used to align reads to human databases, specifically miRBase v22, piRBase v1, and hg38. The outputs of the aligner (Bowtie1) are BAM files, which contain the detected FASTQ sequence and reference sequence to which the detected sequence aligns. The SAMtools idx software tool may be used to tabulate how many detected sequences align to each reference sequence, providing a high-dimensional vector for each FASTQ sample which represents the abundance of each reference RNA in the sample. (Each vector is comprised of many components, each of which represents an RNA abundance.) Thus, nucleotide sequences are transformed into counts of known human miRNAs and piRNAs.

Sequences that do not align to hg38 are then aligned to the NCBI microbial database using k-SLAM. K-SLAM creates pseudo-assemblies of the detected RNA sequences, which are then compared to known microbial sequences and assigned to microbial genes, which are then quantified to microbial identity (eg, genus & species) and activity (eg, metabolic pathway).

These abundances of human short non-coding RNAs, microbial taxa, and metabolic pathways affected by the microbial taxa are then normalized using standard short RNA normalization methods and mathematical adjustments. These include normalizing by the total sum of each RNA category per sample, centering each RNA across samples to 0, and scaling by dividing each RNA by the standard deviation across samples.

As each reference database includes thousands or tens of thousands of reference RNAs, microbes, or cellular pathways, statistical and machine learning feature selection methods are used to reduce the number of potential RNA candidates. Specifically, information theory, random forests, and prototype supervised classification models are used to identify candidate features within subsets of data. Features which are reliably selected across multiple cross-validation splits and feature selection methods comprise the Master Panel of input features.

Features within the Master Panel are ranked using the variable importance within stochastic gradient boosted linear logistic regression machines. Features with high importance are then used as inputs to radial kernel support vector machines, which are used to classify saliva. samples as from ASD or non-ASD children, based on the highly ranked RNA and patient features. In this exemplary application, the features in FIG. 14 are used as the molecular test panel.

Patient features include age, sex, pregnancy or birth complications, body mass index (BMI), gastrointestinal disturbances, and sleep problems. By including these key features, the SVM model identifies different RNA patterns within patient clusters. The output of the SVM model is both a sign (side of the decision boundary) and magnitude (distance from the decision boundary). Thus, each sample can be positioned relative to the decision boundary and assigned a class (ASD or non-ASD) and probability (relative distance from the boundary, as scaled by Platt calibration). In other words, the test model determines the distance from and side of the decision boundary of the patient's test panel sample. This distance of similarity is then translated into a probability that the patient has ASD.

### Results for Operation of the Production Model

A non-limiting exemplary production model is configured to differentiate between young children with autism spectrum disorder (ASD) and other children, either typically developing (JD) or children with developmental delays (DD). The average age of diagnosis in the U.S. is approximately 4 years old, yet studies suggest that early intervention for ASD, before age 2, leads to the best long term prognosis for children with ASD. During the development of this exemplary production model, a sample included children 18 to 83 months (1.5 to 6 years) in order to provide clinical utility aiding in the early childhood diagnostic process.

Prior to operation of the production model, a saliva swab and short online questionnaire are performed and, using the disclosed machine learning procedure classifies the microbiome and non-coding human RNA content in the child's saliva. in particular, each saliva swab is sent to a lab (for example, Admera Health) for RNA extraction and sequencing, and then bioinformatics processing is performed to quantify the amount of 30,000 RNAs found in the saliva. The machine learning procedure identified a panel of 32 RNA features, which are combined with information about the child (age, sex, BMI, etc) to provide a probability that the child will receive a diagnosis of ASD.

The panel includes human microRNAs, piRNAs, microbial species, genera, and RNA activity. MicroRNAs and piRNAs are epigenetic molecules that regulate how active specific genes are. Microbes are known to interact with the brain. The saliva represents both a window into the functioning of the brain, and the microbiome and its relationship with brain health. By quantifying the RNAs found in the mouth, the machine learning procedure identified patterns of RNAs that are useful in differentiating children with ASD from those without.

The panel of 32 RNA features includes 13 miRNAs, 4 piRNAs, 11 microbes, and 4 microbial pathways. These features, adjusted for age, sex, and other medical features, are used in the machine learning procedure to provide a probability that a child will be diagnosed with ASD.

The production model then provides a probability that the child will receive a diagnosis of ASD.

As indicated in the Table below, the study population is representative of children receiving diagnoses of ASD: ages 18 to 83 months, 74% male, with a mixed history of ADHD, sleep problems, GI issues, and other comorbid factors. Children participating in the study represent diverse ethnicities and geographic backgrounds.

| Population characteristics | Total | | ASD | | DD | | TD | |
|---|---|---|---|---|---|---|---|---|
| Children # (%) | 692 | (100%) | 383 (55%) | | 121 | (17%) | 188 | (27%) |
| Male/Female # | 514/178 | | 313/70 | | 86/35 | | 115/73 | |
| % | 74%/26% | | 82%/18% | | 71%/29% | | 61%/39% | |
| Age (months) range | 18-83 | | 20-83 | | 19-83 | | 18-83 | |
| Mean ± SD | 47.5 ± 16.6 | | 48.5 ± 16.4 | | 45.6 ± 14.6 | | 46.5 ± 18.0 | |
| BMI range | 12-40 | | 12-35 | | 12-36 | | 13-40 | |
| Mean ± SD | 16.9 ± 2.8 | | 16.9 ± 2.6 | | 17.1 ± 2.9 | | 16.8 ± 3.0 | |
| ADHD # (%) | 57 | (8%) | 39 | (10%) | 14 | (12%) | 4 | (2%) |
| Asthma | 69 | (10%) | 37 | (10%) | 16 | (13%) | 16 | (9%) |
| Gastrointestinal Issues | 196 | (28%) | 137 | (36%) | 39 | (32%) | 20 | (11%) |
| Sleep Issues | 263 | (38%) | 181 | (47%) | 50 | (41%) | 32 | (17%) |
| Race - White - # (%) | 535 | (77%) | 283 | (74%) | 93 | (77%) | 159 | (85%) |
| African American | 70 | (10%) | 44 | (11%) | 16 | (13%) | 10 | (5%) |
| Hispanic | 66 | (9.5%) | 47 | (12%) | 8 | (7%) | 11 | (6%) |

In children with consensus diagnoses, the production model was found to be highly accurate in identifying children with ASD and children who are typically developing. As expected, the production model tends to give high values to children with ASD and lower values to ID children. In this operation, children who received a score below 25% were most likely typically developing, and most children who received a score above 67% were likely to have ASD.

### Exemplary Hardware

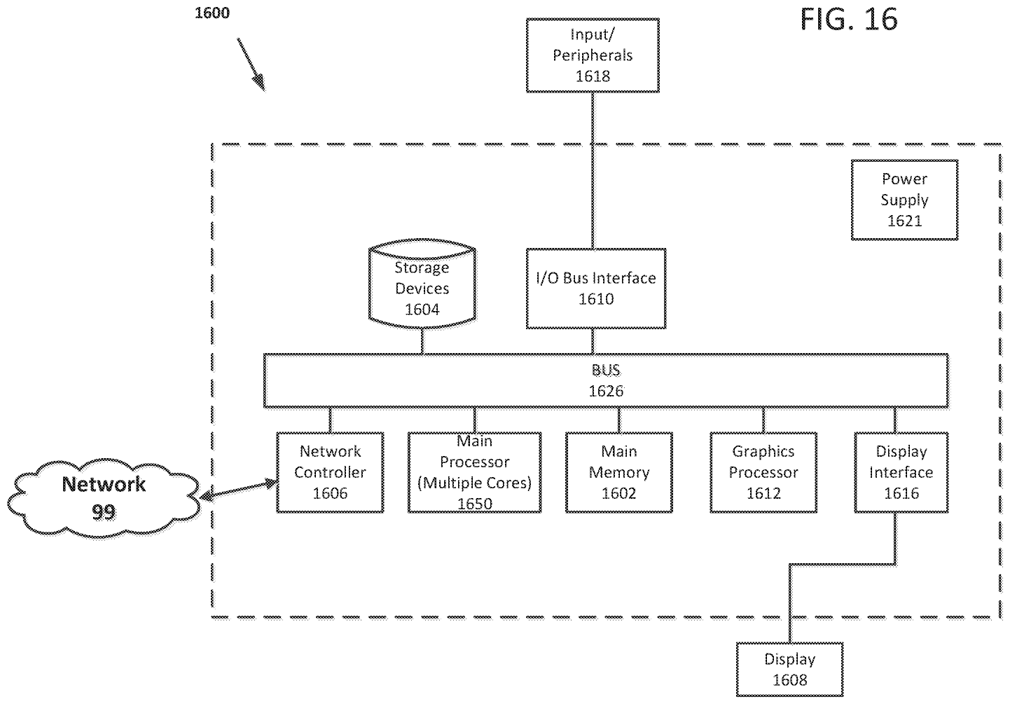

FIG. 16 is a block diagram illustrating an example computer system for implementing the machine learning method according to an exemplary aspect of the disclosure. The computer system may be at least one server or workstation running a server operating system, for example Windows Server, a version of Unix OS, or Mac OS Server, or may be a network of hundreds of computers in a data center providing virtual operating system environments. The computer system 1600 for a server, workstation or networked computers may include one or more processing cores 1650 and one or more graphics processors (GPU) 1612. including one or more processing cores. In an exemplary non-limiting embodiment, the main processing circuitry is an Intel Core i7 and the graphics processing circuitry is the Nvidia Geforce GTX 960 graphics card. The one or more graphics processing cores 1612 may perform many of the mathematical operations of the above machine learning method. The main processing circuitry, graphics processing circuitry, bus and various memory modules that perform each of the functions of the described embodiments may together constitute processing circuitry for implementing the present invention. In some embodiments, processing circuitry may include a programmed processor, as a processor includes circuitry. Processing circuitry may also include devices such as an application specific integrated circuit (ASIC) and circuit components arranged to perform the recited functions. In some embodiments, the processing circuitry may be a specialized circuit for performing artificial neural network algorithms.

The computer system 1600 for a server, workstation or networked computer generally includes main memory 1602, typically random access memory RAM, which contains the software being executed by the processing cores 1650 and graphics processor 1612, as well as a non-volatile storage device 1604 for storing data and the software programs. Several interfaces for interacting with the computer system 1600 may be provided, including an I/O Bus Interface 1610, Input/Peripherals 1618 such as a keyboard, touch pad, mouse, Display interface 1616 and one or more Displays 1608, and a Network Controller 1606 to enable wired or wireless communication through a network 99. The interfaces, memory and processors may communicate over the system bus 1626. The computer system 1600 includes a power supply 1621, which may be a redundant power supply.

Numerous modifications and variations are possible in light of the above teachings. It is therefore to be understood that within the scope of the appended claims, the invention may be practiced otherwise than as specifically described herein.

The various elements, features and processes described herein may be used independently of one another, or may be combined in various ways. All possible combinations and subcombinations are intended to fall within the scope of this disclosure. Further, nothing in the foregoing description is intended to imply that any particular feature, element, component, characteristic, step, module, method, process, task, or block is necessary or indispensable. The example systems and components described herein may be configured differently than described. For example, elements or components may be added to, removed from, or rearranged compared to the disclosed examples.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

The above disclosure also encompasses the embodiments listed below.

(1) A machine learning classifier that diagnoses autism spectrum disorder (ASD), includes processing circuitry that transforms data obtained from a patient medical history and a patient's saliva into data that correspond to a test panel of features, the data for the features including human microtranscriptome and microbial transcriptome data, wherein the transcriptome data are associated with respective RNA categories for ASD; and classifies the transformed data by applying the data to the processing circuitry that has been trained to detect ASD using training data associated with the features of the test panel. The trained processing circuitry includes vectors that define a classification boundary.

(2) The machine learning classifier of feature (1), in which the trained processing circuitry is a support vector machine and the vectors that define the classification boundary are support vectors.

(3) The machine learning classifier of features (1) or (2), in which the trained processing circuitry predicts a probability of ASD based on results of the classifying.

(4) The machine learning classifier of any of features (1) to (3), in which the trained processing circuitry is a deep learning system that continues to learn based on additional transcriptome data.

(5) The machine learning classifier of any of features (1) to (4), in which the processing circuitry transforms the data into data that corresponds to the test panel which includes features of at least one micro RNA selected from the group consisting of hsa-mir-146a, hsa-mir-146b, hsa-miR-92a-3p, hsa-miR-106-5p, hsa-miR-3916, hsa-mir-10a, hsa-miR-378a-3p, hsa-miR-125a-5p, hsa-miR146b-5p, hsa-miR-361-5p, hsa-mir-410, hsa-mir-4461 hsa-miR-15a-5p hsa-miR-6763-3p, hsa-miR-196a-5p, hsa-miR-4668-5p, hsa-miR-378d, hsa-miR-142-3p, hsa-mir-30c-1, hsa-mir-101-2, hsa-mir-151a, hsa-miR-125b-2-3p, hsa-mir-148a-5p, hsa-mir-548I, hsa-miR-98-5p, hsa-miR-8065, hsa-mir-378d-1, hsa-let-7f-1, hsa-let-7d-3p, hsa-let-7a-2, hsa-let-7f-2, hsa-let-7f-5p, hsa-mir-106a, hsa-mir-107, hsa-miR-10b-5p, hsa-miR-1244, hsa-miR-125a-5p, hsa-mir-1268a, hsa-miR-146a-5p, hsa-mir-155, hsa-mir-18a, hsa-mir-195, hsa-mir-199a-1, hsa-mir-19a, hsa-miR-218-5p, hsa-mir-29a, hsa-miR-29b-3p, hsa-miR-29c-3p, hsa-miR-3135b, hsa-mir-3182, hsa-mir-3665, hsa-mir-374a, hsa-mir-421, hsa-mir-4284, hsa-miR-4436b-3p, hsa-miR-4698, hsa-mir-4763, hsa-mir-4798, hsa-mir-502, hsa-miR-515-5p, hsa-mir-5572, hsa-tniR-6724-5p, hsa-mir-6739, hsa-miR-6748-3p, and hsa-miR-6770-5p.

(6) The machine learning classifier of any of features (1) to (5), in which the processing circuitry transforms the data into data that corresponds to the test panel which includes features of at least one piRNA selected from the group consisting of piR-hsa-15023, piR-hsa.-27400, piR-hsa-9491, piR-hsa-29114, piR-hsa-6463, piR-hsa-24085, ,piR-hsa-12423, piR-hsa-24684, piR-hsa-3405, piR-hsa-324, piR-hsa-18905, piR-hsa-23248, piR-hsa-28223, piR-hsa-28400, piR-hsa-1177, piR-hsa-26592, piR-hsa-11361, piR-hsa-26131, piR-hsa-27133, piR-hsa-27134, R-hsa-27282, and piR-hsa-27728.

(7) The machine learning classifier of any of features (1) to (6), in which the processing circuitry transforms the data into data that corresponds to the test panel which includes features of at least one ribosomal RNA selected from the group consisting of RNA5S, MTRNR2L4, and MTRNR2L8.

(8) The machine learning classifier of any of features (1) to (7), in which the processing circuitry transforms the data into data that corresponds to the test panel which includes features of at least one small nucleolar RNA selected from the group consisting of SNORD118, SNORD29, SNORD53B, SNORD68, SNORD20, SNORD41, SNORD30, SNORD34, SNORD110, SNORD28, SNORD45B, and SNORD92.

(9). The machine learning classifier of any of features (1) to (8), in which the processing circuitry transforms the data into data that corresponds to the test panel which includes features of at least one long non-coding RNA.

(10) The machine learning classifier of any of features (1) to (9), in which the processing circuitry transforms the data into data that corresponds to the test panel which includes features of at least one microbe selected from the group consisting of Streptococcus gallolyticus subsp. gallolyticus DSM 16831, Yarrowia lipolytica CLIB122, Clostridiales, Oenococcus oeni PSU-1, Fusarium, Alphaproteobacteria, Lactobacillus fermentum, Corynebacterium uterequi, Ottowia sp. oral taxon 894, Pasteurella multocida subsp. multocida OH4807, Leadbetterella byssophila DSM 17132, Staphylococcus, Rothia, Cryptococcus gattii WM276, Neisseriaceae, Rothia dentocariosa ATCC 17931, Chryseobacterium sp. IHB B 17019, Streptococcus agalactiae CNCTC 10/84, Streptococcus pneumoniae SPNA45, Tsukamurella paurometabola DSM 20162, Streptococcus mutans UA159-FR, Actinomyces oris, Comamonadaceae, Streptococcus halotolerans, Flavobacterium columnare, Streptomyces griseochromogenes, Neisseria, Porphyromonas, Streptococcus salivarius CCHSS3, Megasphaera elsdenii. DSM 20460, Pasteurellaceae, an unclassified Burkholderiales,

Arthrobacter, Dickeya, Jeotgallibacillus, Kocuria, Leuconostoc, Lysinibacillus, Maribacter, Methylophilus, Mycobacterium, Ottowia, Trichormus.

(11) The machine learning classifier of any of features (1) to (10), in which the data from the patient's medical history corresponds to categorical patient features and numerical patient features. The transformation processing circuitry projects the categorical patient features onto principal components.

(12) The machine learning classifier of feature (11), in which the processing circuitry transforms the data into data that corresponds to the test panel which includes features of seven of the patient data principal components and patient age; micro RNAs including: hsa-mir-146a, hsa-mir-146b, hsa-miR-92a-3p, hsa-miR-106-5p, hsa-miR-3916, hsa-miR-10a, hsa-miR-378a-3p, hsa-miR-125a-5p, hsa-miR146b-5p, hsa-miR-361-5p, hsa-mir-410; piRNAs including: piR-hsa-15023, piR-hsa-27400, piR-hsa-9491, piR-hsa-29114, piR-hsa-6463, piR-hsa-24085, piR-hsa-12423, piR-hsa-24684; small nucleolar RNA including: SNORD118; and microbes including: Streptococcus gallolyticus subsp. gallolyticus DSM 16831, Yarrowia lipolytica CLIB122, Clostridiales, Oenococcus oeni PSU-1, Fusarium, Alphaproteobacteria, Lactobacillus fermentum, Corynebacterium uterequi, Ottowia sp. oral taxon 894, Pasteurella multocida subsp. multocida OH4807, Leadbetterella byssophila DSM 17132, Staphylococcus.

(13) The machine learning classifier of feature (11), in which the test panel includes features of seven of the patient data principal components, patient age, and patient sex; micro RNAs including: hsa-let-7a-2, hsa-miR-10b-5p, hsa-miR-125a-5p, hsa-miR-125b-2-3p, hsa-miR-142-3p, hsa-miR-146a-5p, hsa-miR-218-5p, hsa-mir-378d-1, hsa-mir-410, hsa-mir-421, hsa-mir-4284, hsa-miR-4698, hsa-mir-4798, hsa-miR-515-5p, hsa-mir-5572, hsa-miR-6748-3p; piRNAs including: piR-hsa-12423, piR-hsa-15023, piR-hsa-18905, piR-hsa-23638, piR-hsa-24684, piR-hsa-27133, piR-hsa-324, piR-hsa-9491; long nucleolar RNA; microbes including: Actinomyces, Arthrobacter, Jeotgalibacillus, Leadbetterelia, Leuconostoc, Mycobacterium, Ottowia, Saccharomyces; and a microbial activity including: K00520, K14221, K01591, K02111, K14255, K1432, K00133, K03111.

(14) The machine learning classifier of feature (1), in which the test panel of features and the vectors that define the classification boundary are determined by the processing circuitry by fitting a predictive model with an increasing number of features in a Master Panel of features in ranked order until a predictive performance reaches a plateau.

(15) The machine learning classifier of feature (14), in which the predictive model is a support vector machine model.

(16) The machine learning classifier of features (14) or (15), in which the predictive model is a support vector machine model with radial kernel.

(17) The machine learning classifier of any of features (14) to (16), in which the data from the patient's medical history corresponds to categorical patient features and numerical patient features. The transformation processing circuitry projects the categorical patient features onto principal components. The Master Panel includes features of nine of the patient data principal components and patient age; micro RNAs including: hsa-mir-146a, hsa-mir-146b, hsa-miR-92a-3p, hsa-miR-106-5p, hsa-miR-3916, hsa-mir-10a, hsa-miR-378a-3p, hsa-miR-125a-5p, hsa-miR146b-5p, hsa-miR-361-5p, hsa-mir-410, hsa-mir-4461, hsa-miR-15a-5p, hsa-miR-6763-3p, hsa-miR-196a-5p, hsa-miR-4668-5p, hsa-miR-378d, hsa-miR-142-3p, hsa-mir-30c-1, hsa-mir-101-2, hsa-mir-151a, hsa-milk-125b-2-3p, hsa-mir-148a-5p, hsa-mir-548I, hsa-miR-98-5p, hsa-miR-8065, hsa-mir-378d-1, hsa-let-7f-1, and hsa-let-7d-3p; piRNAs including: piR-hsa-15023, piR-hsa-27400, piR-hsa-9491, piR-hsa-29114, piR-hsa-6463, piR-hsa-24085, piR-hsa-12423, piR-hsa-24684, piR-hsa-3405, piR-hsa-324, piR-hsa-18905, piR-hsa-23248, piR-hsa-28223, piR-hsa-28400, piR-hsa-1177, and piR-hsa-26592; small nucleolar RNAs including: SNORD118, SNORD29, SNORD53B, SNORD68, SNORD20, SNORD41, SNORD30, and SNORD34; ribosomal RNAs including: RNASS, MTRNR2L4, and MTRNR2L8; long non-coding RN A including: LOC730338, microbes including: Streptococcus gallolyticus subsp. gallolyticus DSM 16831, Yarrowia lipolytica CLIB122, Clostridiales, Oenococcus oeni PSU-1, Fusarium, Alphaproteobacteria, Lactobacillus fermentum, Corynebacterium uterequi, Ottowia sp. oral taxon 894, Pasteurella multocida subsp. muitocida OH4807, Leadbetterella byssophila DSM 17132, Staphylococcus, Rothia, Cryptococcus gattii WM276, Neissedaceae, Rothia dentocariosa ATCC 17931, Chryseobacterium sp. IHB B 17019, Streptococcus agalactiae CNCTC 10/84, Streptococcus pneumoniae SPNA45, Tsukamurella paurometabola DSM 20162, Streptococcus mutans UA159-FR, Actinomyces oris, Comamonadaceae, Streptococcus halotolerans, Flavobacterium columnare, Streptomyces griseochromogenes, Neisseria, Porphyromonas, Streptococcus salivarius CCHSS3, Megasphaera elsdenii DSM 20460, Pasteurellaceae, and an unclassified Burkholderiales.

(18) The machine learning classifier of any of features (14) to (17), in which the processing circuitry determines the Test Panel of features which includes micro RNAs including: hsa_let 7_d_5p, hsa_let_7g_5p, hsa-Mir_101_3p, hsa-miR_1307_5p, hsa_miR_142_5p, hsa_miR_151a_3p, hsa_miR_15a_5p, hsa_miR_10_3p, hsa_miR_28_3p, hsa_miR_29a_3p, hsa_miR_3074_5p, hsa_miR_374a_5p, hsa_miR_92a_3p; piRNAs including: hsa-piRNA_3499, hsa-piRNA_1433, hsa-piRNA_9843, hsa-piRNA_2533; microbes including: Actinomyces meyeri, Eubacterium, Kocuria flava, Kocuria rhizophila, Kocuria turfanensis, Lactobacillus fermentum, Lysinibacillus sphaericus, Micrococcus luteus, Ottowia, Rothia dentocariosa, Streptococcus dysgalactiae; a microbial activity including: K01867, K02005, K02795, K19972.

(19) A classification machine learning system, includes a data input device that receives as inputs human microtranscriptome and microbial transcriptome data, wherein the transcriptome data are associated with respective RNA categories for a target medical condition; processor circuitry that transforms a plurality of features into an ideal form, determines and ranks each transformed feature from the human microtranscriptome and microbial transcriptome data in terms of predictive power relative to similar features, selects top ranked transformed features from each RNA category, and calculates a joint ranking across all the transcriptome data; the processor circuitry that learns to detect the target medical condition by fitting a predictive model with an increasing number of features from the joint data in ranked order until predictive performance reaches a plateau, sets the features as a test panel, and sets a test model for the target medical condition based on patterns of the test panel features.

(20) The classification machine learning system of feature (19), in which the data input device receives categories of the microtranscriptome data which include one or more of mature microRNA, precursor microRNA, piRNA, snoRNA, ribosomal RNA, long non-coding RNA, and microbes identified by RNA.

(21) The classification machine learning system of features (191 or (20), in which the processing circuitry transforms the features which include RNA derived from saliva via RNA sequencing and microbial taxa identified by RNA derived from the saliva.

(22) The classification machine learning system of any of features (19) to (21), in which the data input device receives the input data which includes patient data extracted from surveys and patient charts. The processor circuitry modifies the rank of specific features that vary depending on the patient data.

(23) The classification machine learning system of feature (22), in which the processing circuitry transforms the features including patient data that varies based on circadian patient data, including one or more of time of collection of saliva sample, time since last meal, time since teeth hygiene treatment.

(24) The classification machine learning system of any of features (19) to (23), in which the processor circuitry includes a stochastic gradient boosting machine circuitry that increases prediction accuracy for each feature type information identified with the categories, ranks each feature type information in order of prediction performance, and selects the top features within each category.

(25) The classification machine learning system of feature (24), in which the stochastic gradient boosting machine is a random forest variant of a stochastic gradient boosting logistic regression machine.

(26) The classification machine learning system of any of features (19) to (25), in which the processor circuitry includes a support vector machine.

(27) The classification machine learning system of any of features (19) to (26), in which the data input device receives the human data and microbial data that are specific to the target medical condition.

(28) The classification machine learning system of feature (27), in which the target medical condition is a condition from the group consisting of autism spectrum disorder, Parkinson's disease, and traumatic brain injury.

(9) The classification machine learning system of any of features (19), in which the data input device receives the genetic data which includes other biomarkers.

(30) The classification machine learning system of feature (22), in which the data input device receives the patient data which includes one or more of time of day, body mass index, age, weight, geographical region of residence at a time that a biological sample is provided by the patient for purposes of obtaining the genetic data.

(31) The classification machine learning system of any of features (19) to (30), in which the data input device receives the human microtranscriptome data which includes nucleotide sequences and a count for each sequence indicating abundance in a biological sample.

(32) A method performed by a machine learning system, the machine learning system including a data input device, and processing circuitry, the method includes receiving as inputs human microtranscriptome and microbial transcriptome data via the data input device, wherein the transcriptome data are associated with respective RNA categories for a target medical condition; transforming a plurality of features into an ideal form; determining and ranking via the processor circuitry each transformed feature from the human microtranscriptome and microbial transcriptome data in terms of predictive power relative to similar features, selects top ranked transformed features from each RNA category, and calculates a joint ranting across all the transcriptome data; learning to detect a target medical condition by fitting a predictive model with an increasing number of features from the joint data in ranked order until predictive performance reaches a plateau; setting the features included as a test panel; and setting a test model for the target medical condition based on patterns of the test panel features.

(33) The method of feature (32), in which the receiving includes receiving categories of the microtranscriptome data which include one or more of mature microRNA, precursor microRNA, piRNA, snoRNA, ribosomal RNA, long non-coding RNA, and identified by RNA.

(34) The method of features (32) or (33), in which the receiving includes receiving the features which include RNA derived from saliva via RNA sequencing and microbial taxa identified by RNA derived from the saliva.

(35) The method of any of features (32) to (34), further includes receiving patient data extracted from surveys and patient charts; and modifying, by the processing circuitry, the rank of specific features that vary depending on the patient data.

(36) The method of feature (35), in which the receiving includes receiving the patient data that vary based on circadian patient data, including one or more of time of collection of saliva sample, time since last meal, time since teeth hygiene treatment.

(37) The method of feature (32), in which the target medical condition is a condition from the group consisting of autism spectrum. disorder, Parkinson's disease, and traumatic brain injury.

(38) A non-transitory computer-readable storage medium storing program code, which when executed by a machine learning system, the machine learning system including a data input device, and processor circuitry, the program code performs a method including receiving as inputs human microtranscriptome and microbial transcriptome data via the data input device, wherein the transcriptome data are associated with respective RNA categories for a target medical condition; transforming a plurality of features into an ideal form; determining and ranking each transformed feature from the human microtranscriptome and microbial transcriptome data in terms of predictive power relative to similar features, selects top ranked transformed features from each RNA category, and calculates a joint ranking across all the transcriptome data; learning to detect a target medical condition by fitting a predictive model with an increasing number of features from the joint data in ranked order until predictive performance reaches a plateau; setting the features included as a test panel; and setting a test model for the target medical condition based on patterns of the test panel features.

All publications, patent applications, patents, and other references mentioned herein are incorporated by reference in their entirety. Further, the materials, methods, and examples are illustrative only and are not intended to be limiting, unless otherwise specified.

### LITERATURE

1. Ambros et al. The functions of animal microRNAs, Nature, 431 (7006):350-5 (Sep. 16, 2004), herein incorporated by reference in its entirety.
2. Bartel et al., MicroRNAs: genomics, biogenesis, mechanism, and function, Cell, 116 (2): 281-97 (Jan. 23, 2004), herein incorporated by reference in its entirety.
3. Xu L M, Li J R, Huang Y, Zhao M, Tang X, Wei L. AutismKB: an evidence-based knowledgebase of autism genetics. Nucleic Acids Res 2012;40:D1016-22, herein incorporated by reference in its entirety.
4. Gallo A, Tandon M, Alevizos I, Illei G G. The majority of microRNAs detectable in serum and saliva is concentrated in exosomes. PLOS One 2012;7:e30679, herein incorporated by reference in its entirety.
5. Mulle, J. G., Sharp, W. G., & Cubells, J. F., The gut microbiome: a new frontier in autism research, Current Psychiatry Eeports, 15(2), 337 (2013), herein incorporated by reference in its entirety.

What is claimed is:

1. A machine learning classifier that diagnoses autism spectrum disorder (ASD), comprising:
processing circuitry that
transforms data obtained from a patient medical history and a patient's saliva into data that correspond to a test panel of features, the data for the features including human microtranscriptome and microbial transcriptome data, wherein the transcriptome data are associated with respective RNA categories for ASD; and
classifies the transformed data by applying the data to the processing circuitry that has been trained to detect ASD using training data associated with the features of the test panel,
wherein the trained processing circuitry includes vectors that define a classification boundary.

2. The machine learning classifier of claim 1, wherein the trained processing circuitry is a support vector machine and the vectors that define the classification boundary are support vectors.

3. The machine learning classifier of claim 1, wherein the trained processing circuitry predicts a probability of ASD based on results of the classifying.

4. The machine learning classifier of claim 1, wherein the trained processing circuitry is a deep learning system that continues to learn based on additional transcriptome data.

5. The machine learning classifier of claim 1, wherein the processing circuitry transforms the data into data that corresponds to the test panel of features which includes at least one micro RNA selected from the group consisting of hsa-mir-146a, hsa-mir-146b, hsa-miR-92a-3p, hsa-miR-106-5p, hsa-miR-3916, hsa-mir-10a, hsa-miR-378a-3p, hsa-miR-125a-5p, hsa-miR146b-5p, hsa-miR-361-5p, hsa-mir-410, hsa-mir-4461, hsa-miR-15a-5p, hsa-miR-6763-3p, hsa-miR-196a-5p, hsa-miR-4668-5p, hsa-miR-378d, hsa-miR-142-3p, hsa-mir-30c-1, hsa-mir-101-2, hsa-mir-151a, hsa-miR-125b-2-3p, hsa-mir-148a-5p, hsa-mir-548I, hsa-miR-98-5p, hsa-miR-8065, hsa-mir-378d-1, hsa-let-7f-1, hsa-let-7d-3p,
hsa-let-7a-2, hsa-let-7f-2, hsa-let-7f-5p, hsa-mir-106a, hsa-mir-107, hsa-miR-10b-5p, hsa-miR-1244, hsa-miR-125a-5p, hsa-mir-1268a, hsa-miR-146a-5p, hsa-mir-155, hsa-mir-18a, hsa-mir-195, hsa-mir-199a-1, hsa-mir-19a, hsa-miR-218-5p, hsa-mir-29a, hsa-miR-29b-3p, hsa-miR-29c-3p, hsa-miR-3135b, hsa-mir-3182, hsa-mir-3665, hsa-mir-374a, hsa-mir-421, hsa-mir-4284, hsa-miR-4436b-3p, hsa-miR-4698, hsa-mir-4763, hsa-mir-4798, hsa-mir-502, hsa-miR-515-5p, hsa-mir-5572, hsa-miR-6724-5p, hsa-mir-6739, hsa-miR-6748-3p, and hsa-miR-6770-5p.

6. The machine learning classifier of claim 1, wherein the processing circuitry transforms the data into data that corresponds to the test panel of features which includes at least one piRNA selected from the group consisting of piR-hsa-15023, piR-hsa-27400, piR-hsa-9491, piR-hsa-29114, piR-hsa-6463, piR-hsa-24085, piR-hsa-12423, piR-hsa-24684, piR-hsa-3405, piR-hsa-324, piR-hsa-18905, piR-hsa-23248, piR-hsa-28223, piR-hsa-28400, piR-hsa-1177, piR hsa-26592,
piR-hsa-1136, R-hsa-26131, piR-hsa-27133, piR-hsa-27134, piR-hsa-27282, and piR-hsa-27728.

7. The machine learning classifier of claim 1, wherein the processing circuitry transforms the data into data that corresponds to the test panel of features which includes at least one ribosomal RNA selected from the group consisting of RNA5S, MTRNR2L4, and MTRNR2L8.

8. The machine learning classifier of claim 1, wherein the processing circuitry transforms the data into data that corresponds to the test panel of features which includes at least one small nucleolar RNA selected from the group consisting of SNORD118, SNORD29, SNORD53B, SNORD68, SNORD20, SNORD41, SNORD30, SNORD34, SNORD110, SNORD28, SNORD45B, and SNORD92.

9. The machine learning classifier of claim 1, wherein the processing circuitry transforms the data into data that corresponds to the test panel which includes features of at least one long non-coding RNA.

10. The machine learning classifier of claim 1, wherein the processing circuitry transforms the data into data that corresponds to the test panel of features which includes at least one microbe selected from the group consisting of Streptococcus gallolyticus subsp. gallolyticus DSM 16831, Yarrowia lipolytica CLIB122, Clostridiales, Oenococcus oeni PSU-1, Fusarium, Alphaproteobacteria, Lactobacillus fermentum, Corynebacterium uterequi, Ottowia sp. oral taxon 894, Pasteurella multocida subsp. multocida OH4807, Leadbetterella byssophila DSM 17132, Staphylococcus, Rothia, Cryptococcus gattii WM276, Neisseriaceae, Rothia dentocariosa ATCC 17931, Chryseobacterium sp. IHB B 17019, Streptococcus agalactiae CNCTC 10/84, Streptococcus pneumoniae SPNA45, Tsukamurella paurometabola DSM 20162, Streptococcus mutans UA159-FR, Actinomyces oris, Comamonadaceae, Streptococcus halotolerans, Flavobacterium columnare, Streptomyces griseochromogenes, Neisseria, Porphyromonas, Streptococcus salivarius CCHSS3, Megasphaera elsdenii DSM 20460, Pasteurellaceae, an unclassified Burkholderiales, Arthrobacter, Dickeya, Jeotgalibacillus, Kocuria, Leuconostoc, Lysinibacillus, Maribacter, Methylophilus, Mycobacterium, Ottowia, Trichormus.

11. The machine learning classifier of claim 1, wherein the data from the patient's medical history corresponds to categorical patient features and numerical patient features,
wherein the processing circuitry projects the categorical patient features onto principal components.

12. The machine learning classifier of claim 11, wherein the processing circuitry transforms the data into data that corresponds to the test panel of features which comprises:
seven of the patient data principal components and patient age;
micro RNAs including: hsa-mir-146a, hsa-mir-146b, hsa-miR-92a-3p, hsa-miR-106-5p, hsa-miR-3916, hsa-mir-10a, hsa-miR-378a-3p, hsa-miR-125a-5p, hsa-miR146b-5p, hsa-miR-361-5p, hsa-mir-410;
piRNAs including: piR-hsa-15023, piR-hsa-27400, piR-hsa-9491, piR-hsa-29114, piR-hsa-6463, piR-hsa-24085, piR-hsa-12423, piR-hsa-24684;
small nucleolar RNA including: SNORD118; and
microbes including: Streptococcus gallolyticus subsp. gallolyticus DSM 16831, Yarrowia lipolytica CLIF3122, Clostridiales, Oenococcus oeni PSU-1, Fusarium, Alphaproteobacteria, Lactobacillus fermentum, Corynebacterium uterequi, Ottowia sp. oral taxon 894, Pasteurella multocida subsp. multocida OH4807, Leadbetterella byssophila DSM 17132, Staphylococcus.

13. The machine learning classifier of claim 11, wherein the processing circuitry transforms the data into data that corresponds to the test panel of features which comprises:
seven of the patient data principal components, patient age, and patient sex;
micro RNAs including: hsa-let-7a-2, hsa-miR-10b-5p, hsa-miR-125a-5p, hsa-miR-125b-2-3p, hsa-miR-142-3p, hsa-miR-146a-5p, hsa-miR-218-5p, hsa-mir-378d-1, hsa-mir-410, hsa-mir-421, hsa-mir-4284, hsa-miR-4698, hsa-mir-4798, hsa-miR-515-5p, hsa-mir-5572, hsa-miR-6748-3p;
piRNAs including: piR-hsa-12423, piR-hsa-15023, piR-hsa-18905, piR-hsa-23638, piR-hsa-24684, piR-hsa-27133, piR-hsa-324, piR-hsa-9491;
long nucleolar RNA;
microbes including: Actinomyces, Arthrobacter, Jeotgalibacillus, Leadbetterella, Leuconostoc, Mycobacterium, Ottowia, Saccharomyces; and
a microbial activity including: K00520, K14221, K01591, K02111, K14255, K1432, K00133, K03111.

14. The machine learning classifier of claim 1, wherein the test panel of features and the vectors that define the classification boundary are determined by the processing circuitry by fitting a predictive model with an increasing number of features in a Master Panel of features in ranked order until a predictive performance reaches a plateau.

15. The machine learning classifier of claim 14, wherein the predictive model is a support machine model.

16. The machine learning classifier of claim 14, wherein the predictive model is a support vector machine model with radial kernel.

17. The machine learning classifier of claim 14, wherein the data from the patient's medical history corresponds to categorical patient features and numerical patient features,
wherein the processing circuitry projects the categorical patient features onto principal components,
wherein the processing circuitry transforms the data into data that corresponds to the Master Panel of features which comprises:
nine of the patient data principal components and patient age;
micro RNAs including: hsa-mir-146a, hsa-mir-146b, hsa-miR-92a-3p, hsa-miR-106-5p, hsa-miR-3916, hsa-mir-10a, hsa-miR-378a-3p, hsa-miR-125a-5p, hsa-miR146b-5p, hsa-miR-361-5p, hsa-mir-410, hsa-mir-4461 hsa-miR-15a-5p, hsa-miR-6763-3p, hsa-miR-196a-5p, hsa-miR-4668-5p, hsa-miR-378d, hsa-miR-142-3p, hsa-mir-30c-1, hsa-mir-101-2, hsa-mir-151a, hsa-miR-125b-2-3p, hsa-mir-148a-5p, hsa-mir-548I, hsa-miR-98-5p, hsa-miR-8065, hsa-mir-378d-1, hsa-let-7f-1, and hsa-let-7d-3p;
piRNAs including: piR-hsa-15023, piR-hsa-27400, piR-hsa-9491, piR-hsa-29114, piR-hsa-6463, piR-hsa-24085, piR-hsa-12423, piR-hsa-24684, piR-hsa-3405, piR-hsa-324, piR-hsa-18905, piR-hsa-23248, piR-hsa-28223, piR-hsa-28400, piR-hsa-1177, and piR-hsa-26592;
small nucleolar RNAs including: SNORD118, SNORD29, SNORD53B, SNORD68, SNORD20, SNORD41, SNORD30, and SNORD34;
ribosomal RNAs including: RNA5S, MTRNR2L4, and MTRNR2L8;
long non-coding RNA including: LOC730338;
microbes including: Streptococcus gallolyticus subsp. gallolyticus DSM 16831, Yarrowia lipolytica CLIB122, Clostridiales, Oenococcus oeni PSU-1, Fusarium, Alphaproteobacteria, Lactobacillus fermentum, Corynebacterium uterequi, Ottowia sp. oral taxon 894, Pasteurella multocida subsp. multocida OH4807, Leadbetterella byssophila DSM 17132, Staphylococcus, Rothia, Cryptococcus gattii WM276, Neisseriaceae, Rothia dentocariosa ATCC 17931, Chryseobacterium sp. IHB B 17019, Streptococcus agalactiae CNCTC 10/84, Streptococcus pneumoniae SPNA45, Tsukamurella paurometabola DSM 20162, Streptococcus mutans UA159-FR, Actinomyces oris, Comamonadaceae, Streptococcus halotolerans, Flavobacterium columnare, Streptomyces griseochromogenes, Neisseria, Porphyromonas, Streptococcus salivarius CCHSS3, Megasphaera elsdenii DSM 20460, Pasteurellaceae, and an unclassified Burkholderiales.

18. The machine learning classifier of claim 14, wherein the processing circuitry determines the Test Panel of features which comprises:
micro RNAs including: hsa_let_7d_5p, hsa_let_7g_5p, hsa_miR_101_3p, hsa_miR_1307_5p, hsa_miR_142_5p, hsa_miR_151a_3p, hsa_miR_15a_5p, hsa_miR_210_3p, hsa_miR_28_3p, hsa_miR_29a_3p, hsa_miR_3074_5p, hsa_miR 374a_5p, hsa_miR_92a_3p;
piRNAs including: hsa-piRNA_3499, hsa-piRNA_1433, hsa-piRNA_9843, hsa-piRNA_2533;
microbes including: Actinomyces meyeri, Eubacterium, Kocuria flava, Kocuria rhizophila, Kocuria turfanensis, Lactobacillus fermentum, Lysinibacillus sphaericus, Micrococcus luteus, Ottowia, Rothia dentocariosa, Streptococcus dysgalactiae;
a microbial activity including: K01867, K02005, K02795, K19972.

19. A classification machine learning system, comprising:
a data input device that receives as inputs human microtranscriptome and microbial transcriptome data, wherein the transcriptome data are associated with respective RNA categories for a target medical condition;
processing circuitry that transforms a plurality of features into an ideal form, determines and ranks each transformed feature from the human microtranscriptome and microbial transcriptome data in terms of predictive power relative to similar features, selects top ranked transformed features from each RNA category, and calculates a joint ranking across all the transcriptome data;
the processing circuitry learns to detect the target medical condition by fitting a predictive model with an increasing number of features from the joint data in ranked order until predictive performance reaches a plateau, sets the features as a test panel, and sets a test model for the target medical condition based on patterns of the test panel features.

20. The classification machine learning system of claim 19, wherein the data input device receives the categories of the microtranscriptome data which include one or more of mature microRNA, precursor microRNA, piRNA, snoRNA, ribosomal RNA, long non-coding RNA, and microbes identified by RNA.

21. The classification machine learning system of claim 19, wherein the processing circuitry transforms the features which include RNA derived from saliva via RNA sequencing and microbial taxa identified by RNA derived from the saliva.

22. The classification machine learning system of claim 19, wherein the data input device receives the input data which includes patient data extracted from surveys and patient charts,
wherein the processor circuitry modifies the rank of specific features that vary depending on the patient data.

23. The classification machine learning system of claim 22, wherein the processing circuitry transforms the features including patient data that varies based on circadian patient data, including one or more of time of collection of saliva sample, time since last meal, time since teeth hygiene treatment.

24. The classification machine learning system of claim 19, wherein the processing circuitry includes a stochastic gradient boosting machine circuitry that increases prediction accuracy for each feature type information identified with the categories, ranks each feature type information in order of prediction performance, and selects the top features within each category.

25. The classification machine learning system of claim 24, wherein the stochastic gradient boosting machine is a random forest variant of a stochastic gradient boosting logistic regression machine.

26. The classification machine learning system of claim 19, wherein the processor circuitry includes a support vector machine.

27. The classification machine learning system of claim 19, wherein the data input device receives the human data and microbial data that are specific to the target medical condition.

28. The classification machine learning system of claim 27, wherein the target medical condition is a condition from the group consisting of autism spectrum disorder, Parkinson's disease, and traumatic brain injury.

29. The classification machine learning system of claim 19, wherein the data input device receives the genetic data which includes other biomarkers.

30. The classification machine learning system of claim 22, wherein the data input device receives the patient data which includes one or more of time of day, body mass index, age, weight, geographical region of residence at a time that a biological sample is provided by the patient for purposes of obtaining the genetic data.

31. The classification machine learning system of claim 19, wherein the data input device receives the human microtranscriptome data which includes nucleotide sequences and a count for each sequence indicating abundance in a biological sample.

32. A method performed by a machine learning system, the machine learning system including a data input device, and processor circuitry, the method comprising:
receiving as inputs human microtranscriptome and microbial transcriptome data via the data input device, wherein the transcriptome data are associated with respective RNA categories for a target medical condition;
transforming, by the processing circuitry, a plurality of features into an ideal form;
determining and ranking by the processor circuitry each transformed feature from the human microtranscriptome and microbial transcriptome data in terms of predictive power relative to similar features, selects top ranked transformed features from each RNA category, and calculates a joint ranking across all the transcriptome data;
learning, by the processing circuitry, to detect a target medical condition by fitting a predictive model with an increasing number of features from the joint data in ranked order until predictive performance reaches a plateau;
setting, by the processing circuitry, the features included as a test panel; and
setting, by the processing circuitry, a test model for the target medical condition based on patterns of the test panel features.

33. The method of claim 32, wherein the receiving includes receiving categories of the microtranscriptome data which include one or more of mature microRNA, precursor microRNA, piRNA, snoRNA, ribosomal RNA, long non-coding RNA, and identified by RNA.

34. The method of claim 32, wherein the receiving includes receiving the features which include RNA derived from saliva via RNA sequencing and microbial taxa identified by RNA derived from the saliva.

35. The method of claim 32, further comprising receiving patient data extracted from surveys and patient charts; and
modifying, by the circuitry, the rank of specific features that vary depending on the patient data.

36. The method of claim 35, wherein the receiving includes receiving the patient data that vary based on circadian patient data, including one or more of time of collection of saliva sample, time since last meal, time since teeth hygiene treatment.

37. The method of claim 32, wherein the target medical condition is a condition from the group consisting of autism spectrum disorder, Parkinson's disease, and traumatic brain injury.

38. A non-transitory computer-readable storage medium storing program code, which when executed by a machine learning system, the machine learning system including a data input device, and processor circuitry, the program code performs a method comprising:
receiving as inputs human microtranscriptome and microbial transcriptome data via the data input device, wherein the transcriptome data are associated with respective RNA categories for a target medical condition;
transforming a plurality of features into an ideal form;
determining and ranking each transformed feature from the human microtranscriptome and microbial transcriptome data in terms of predictive power relative to similar features, selects top ranked transformed features from each RNA category, and calculates a joint ranking across all the transcriptome data;
learning to detect a target medical condition by fitting a predictive model with an increasing number of features from the joint data in ranked order until predictive performance reaches a plateau;
setting the features included as a test panel; and
setting a test model for the target medical condition based on patterns of the test panel features.
